(19) United States
(12) Patent Application Publication
(10) Pub. No.: US 2017/0326293 A1
(43) Pub. Date: Nov. 16, 2017

(54) Syringe Infusion Pump

(71) Applicant: The General Hospital Corporation, Boston, MA (US)

(72) Inventors: Nathaniel M. Sims, Milton, MA (US); Eric John Flachbart, Newport Center, VT (US); Duane Edward Allen, Sheffield, VT (US); Benjamin James Chomyn, Fairfield, VT (US); Paul C. Henninge, Burlington, VT (US); Joseph Matthew Pasquence, Plainfield, VT (US); Andrew W. Asack, Barton, VT (US); Michael H. Wollowitz, Chatham, NY (US); Rolf E. Zuk, Monroe, NH (US)

(21) Appl. No.: 15/526,106

(22) PCT Filed: Nov. 12, 2015
(86) PCT No.: PCT/US15/60312

§ 371 Date: May 11, 2017

Related U.S. Application Data
(60) Provisional application No. 62/078,937, filed on Nov. 12, 2014.

(51) Int. Cl. A61M 5/145

(52) U.S. Cl. CPC A61M 5/1458; A61M 5/1456; A61M 2205/502; A61M 2205/332

(57) ABSTRACT

An infusion apparatus for use with a syringe includes a housing enclosing a drivetrain. The drivetrain includes a pinion, spur gears, and a worm gear. One spur gear is arranged to engage with the pinion and another spur gear is arranged to engage with the worm gear. The housing also includes a carriage movable with respect to the housing. A frame on the carriage receives a syringe plunger. A rack on the carriage engages with the pinion to move the carriage parallel to a longitudinal axis of the housing. A pusher assembly of the housing securely engages with the syringe plunger. A motor in the apparatus rotates a worm drive that meshes with the worm gear to drive the drivetrain. A trigger of the apparatus is configured to disengage the worm drive from the worm gear to allow free movement of the carriage relative to the housing.

### CROSS-REFERENCE TO RELATED APPLICATION

This application claims priority to U.S. Provisional Application No. 62/078,937 filed on Nov. 12, 2014, the entire content of which is incorporated herein by reference.

### TECHNICAL FIELD

This disclosure relates to infusion pumps, and more particularly to syringe pumps.

### BACKGROUND

Infusion pumps are used for infusing fluids, which can include drugs or nutrients, into circulatory systems of humans or animals, including life support drugs in critically ill patients. Infusion pumps can be of various types, such as syringe infusion pumps and volumetric infusion pumps. Syringe infusion pumps can be configured to accept standard syringes of various sizes, for example, syringes with volumes ranging from 1.0 cc to 60 cc, made by multiple manufacturers. In some implementations, operations of a syringe pump can be controlled via a motor under the control of a microprocessor. The motor can be connected to a lead screw that advances to push a pushing element against a plunger of the syringe. The pushing element drives the plunger into the body of the syringe, thus dispensing a medical fluid from the syringe into a flexible tubing set connected to the patient's vascular system. In some cases, the infusion pumps can include sensors for determining the size of syringe loaded, the position of the plunger within its travel, whether the plunger is captured by the pushing element, and the driving force needed to push the plunger. The devices can also include encoders or other mechanisms for determining the motor speed. Syringes from different manufacturers are made according to international standards such as ISO 7886-2 to encourage consistency in key parameters such as compliance under pressure (microliters per mmHg) and syringe body length and inner diameter.

In a device that uses a lead screw in the syringe drive, the lead screw translates rotational movement of the screw to linear movement, which in turn drives the syringe plunger typically by a nut or half nut. When a nut is used, disengagement of the drive from the screw is not possible, thereby requiring the user to advance the position of the drive head (i.e., for removing an empty syringe and replacing it with a refilled one) by running the motor. In some cases, this may require additional time to move the drive head into position to install the syringe. When a half nut is used, disengagement is possible, but the additional components required may increase mechanical lash resulting in delays in initiation of fluid flow when an infusion is started, delays in detection of downstream occlusions to flow, and reduced fluid flow consistency or smoothness of medical fluid flow.

The volumetric accuracy of lead screw based pumps can be influenced by imprecision of lead spacing of the lead screw, as well as inaccuracies contributed by the half nut detachment mechanism, linear misalignment of the lead screw to the syringe axis, concentricity of the lead screw journals to the screw axis, pitch error, and other mechanical factors. Lead error can be expressed, for example, in an expected linear travel error over one turn of the screw. This error can be cumulative over the number of lead screw turns required to travel a linear distance. In the application of a syringe pump the number of lead screw turns required to drive a syringe a given distance corresponds to the volume of drug to be delivered. Smaller syringes that require more turns per unit volume of fluid may suffer greater volumetric inaccuracies due to lead error and lead error contributions of the screw/nut interface. However, if larger size syringes (e.g., 60 ml) are used at low flow rates of, for example, less than 1.0 ml/hour, the pump will be operating at or near the limit of movement resolution of the stepper motor, gear reduction, and lead screw pitch performance envelope, with the motor needing to pause for up to several seconds between each motor step.

Accordingly, syringe infusion pumps with lead screw-based plunger driving mechanisms, especially those which use a half-nut to permit manual positioning of the drive head, may present numerous challenges to safe and effective operation by users, particularly when the pumps are operating at low fluid flow rates and are delivering drugs to which a patient's physiology is sensitive. These challenges include: (i) low flow rate operation at or near the limits of movement resolution; (ii) delays in starting and stopping of fluid flow including delay of flow initiation due to unavoidable ‘slack’ in mechanical plunger driving mechanisms, requiring users to adopt ‘manual-priming’ procedures; (iii) flow continuity variance due to, for example, drive mechanism issues and mechanical properties of the syringes, such as “stiction” and syringe body taper; (iv) detection and management of downstream occlusions with available pressure sensing technology, (v) mitigation of unintentional bolus release after the user is alerted to a downstream occlusions; and (vi) “environment-of-care” factors including changing head height, patient respiration, high-resistance vascular access devices, and other factors. Thus, improvements to syringe pump drive mechanisms must successfully address the known problems with lead screw and half nut designs, without inducing adverse performance in other key aspects of syringe pump performance impacting safe and efficacious patient care.

### SUMMARY

The present disclosure is related to syringe infusion pumps that infuse medical fluid contained in a syringe into a patient for treatment. In particular, the present disclosure covers devices and methods in which syringe pumps include a carriage and a main body. The syringe pumps include a rack-and-pinion mechanism to produce a linear actuation motion of the carriage, which secures the syringe plunger relative to the main body, which, in turn, holds the syringe body. During operation, the rack-and-pinion mechanism includes features so that forces external to the drivetrain, such as internal fluid pressure or manual handling, cannot substantially or easily backdrive the drivetrain. The syringe pumps further include a mechanism to disengage a stage of the drivetrain so as to allow a user to manually reposition the carriage relative to the main body. Both the carriage and the main body include features to secure the syringe.

In one aspect, an example of infusion devices includes an infusion apparatus adapted for use with a syringe include a housing enclosing a drivetrain. The housing includes a pinion, and a plurality of spur gears and a worm gear. At least one of the spur gears is arranged to engage with the pinion and one of the spur gears is arranged to engage with the worm gear. The infusion device also includes a carriage movable with respect to the housing and having a first side and a second side opposite the first side. The carriage includes a frame on the first side of the carriage. The frame is configured to receive at least a portion of a plunger of the syringe, and a toothed rack disposed on the second side of the carriage, and extending along a longitudinal axis of the carriage. The rack is configured to engage with the pinion for moving the carriage in a direction parallel to a longitudinal axis of the housing. The carriage also includes a pusher assembly adapted to securely engage with the plunger of the syringe. The apparatus further includes a motor arranged within the housing configured to rotate a worm drive that meshes with the worm gear to drive the drivetrain. The apparatus also includes a release mechanism that includes a release trigger configured to enable the worm drive to be disengaged from the worm gear, thereby enabling free movement of the carriage with respect to the housing in a rearward direction.

In another aspect, this document features a method of dispensing a fluid from a syringe disposed on an infusion pump. The method includes engaging a motor with a drivetrain via a release mechanism configured to enable a worm drive to be engaged to a worm gear of a drivetrain, and controlling a movement of a plunger of the syringe through a body of the syringe using the drivetrain. The drivetrain includes a pinion, and a plurality of spur gears and a worm gear. At least one of the spur gears is arranged to engage with the pinion, and one of the spur gears is arranged to engage with the worm gear. The plunger is disposed on a carriage having a first side and a second side opposite the first side. The carriage includes a frame on the first side of the carriage, configured to receive at least a portion of a plunger of the syringe, and a toothed rack disposed on the second side of the carriage. The toothed rack extends along a longitudinal axis of the carriage. The rack is configured to engage with the pinion for moving the carriage in a direction parallel to a longitudinal axis of the carriage. A pusher assembly is adapted to securely engage with the plunger of the syringe.

In a further aspect, an example of infusion devices includes an infusion apparatus adapted for use with a syringe. The apparatus includes a housing having a drivetrain. The drivetrain includes a pinion, and a gear train including spur gears. The spur gears include a first clutch gear, a second clutch gear arranged to engage with the first clutch gear, and a spur gear arranged to engage with the pinion. The apparatus includes a carriage movable with respect to the housing and having a first side and a second side opposite the first side. The carriage includes a frame on the first side of the carriage and a toothed rack disposed on the second side of the carriage. The frame is configured to receive at least a portion of a plunger of the syringe. The toothed rack extends along a longitudinal axis of the carriage. The rack is configured to engage with the pinion for moving the carriage in a direction parallel to a longitudinal axis of the housing. The carriage further includes a pusher assembly adapted to securely engage with the plunger of the syringe. The infusion apparatus includes a motor arranged within the housing configured to rotate a worm drive to drive the gear train. The infusion apparatus further includes a release mechanism including a release trigger. The release trigger is configured to disengage the second clutch gear from the first clutch gear, thereby enabling movement of the carriage with respect to the housing, the movement being independent of a motion of the worm drive.

Implementations can include one or more of the following features.

In some examples, a motor is arranged within the housing. The motor can be configured to actuate the release mechanism such that the first clutch gear is movable between a first position in which the first clutch gear is engaged to the second clutch gear and a second position in which the first clutch gear is disengaged from the second clutch gear.

In some examples, the first clutch gear is a male clutch gear, the second clutch gear is a female clutch gear, and the male clutch gear, in the first position, engages inner teeth of the female clutch gear.

In some examples, the movable carriage can include a plurality of wheels that allow the carriage to move along tracks arranged on the housing, the tracks disposed along the longitudinal axis of the housing. One or more of the tracks can include an asymmetric v-profile track configured to accept one or more v-profile roller wheels of the carriage. The asymmetric v-profile can include two inclined surfaces joined along a line. An angle of incline of one of the surfaces can be less than an angle of incline of the other surface. The tracks can be configured to accept the plurality of wheels such that a translation motion of the wheels in directions perpendicular to the direction parallel to the length of the rack is constrained.

In some examples, the frame of the carriage can be constructed of material that includes a fiber-reinforced plastic composite.

In some examples, a wheelbase corresponding to a pair of wheels can be substantially equal to one half of a maximum distance traveled by the carriage.

In some examples, the motor can be a stepper motor. One or more of the spur gears can be configured to prevent backlash effects.

In some examples, a secondary motor can be arranged within the housing configured to actuate the release mechanism such that the worm drive rotates from an engaged position in which the worm drive is engaged with the worm gear to a disengaged position in which the worm drive is disengaged from the worm gear.

In some examples, the frame can include a pair of hinged arms configured to hold a body of the syringe onto the frame. The hinged arms can be convex in shape, and counter sprung towards one another. The hinged arms can be configured to hold the syringe on the frame such that a longitudinal axis of the plunger is aligned to a center of the pusher assembly. The hinged arms can be movable relative to the pusher assembly in a direction parallel to the longitudinal axis of the carriage. A spring assembly can be configured to apply a force on each of the hinged arms along the longitudinal axis of the carriage toward the pusher assembly.

In some examples, the apparatus can include a display device operable to display one or more parameters related to a fluid delivered using the infusion apparatus. The display device can be attached to the housing via one or more hinges. The one or more hinges can be positioned to prevent the display device from covering at least a portion of a body of the syringe. The display device can be configured to accept user input related to an operation of the apparatus.

In some examples, the apparatus can include one or more processing devices configured to control operations of the motor. The apparatus can include at least one force sensor configured to provide a feedback signal to the one or more processing devices. The one or more processing devices can be configured to generate a control signal to adjust a speed or a direction of the motor in response to the feedback signal. The at least one force sensor can include a force sensor configured to measure a force exerted by the pusher assembly on the plunger.

In some examples, a trigger button is movable relative to the housing and is configured to actuate the release mechanism such that the first clutch gear translates from a first position in which the first clutch gear is engaged to the second clutch gear to a second position in which the first clutch gear is disengaged from the second clutch gear.

The technologies described herein can provide several advantages. For example, the drive mechanism can allow small amounts of medical fluid to be delivered to a patient with a high degree of volumetric accuracy, with consistency of flow over long periods of time, and with sensitive capability to detect fault conditions. The drive mechanism further includes a worm drive engaging with a worm gear to prevent accidental backdriving.

In addition, the drive mechanism can allow a user to manually position the carriage for rapid syringe loading and unloading. The syringe pumps described herein can include elements that can be produced rapidly and inexpensively using common materials, such as reinforced polymers. Affordable standard electromechanical components can be implemented without disrupting the overall precision of the device. The user can control and monitor the fluid and drug administration process through the interactive display on the syringe pump that receives information from a combination of a controller and sensing systems in the syringe pump.

Unless otherwise defined, all technical and scientific terms used herein have the same meaning as commonly understood by one of ordinary skill in the art to which the subject matter of this disclosure belongs. Although methods and materials similar or equivalent to those described herein can be used in the practice or testing of the implementations described herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages will be apparent from the following detailed description, and from the claims.

### DESCRIPTION OF DRAWINGS

FIG. 1 is a side perspective view of an example of an implementation of a syringe pump described herein.

FIGS. 2A and 2B are the top and bottom perspective views, respectively, of an example of a carriage of the syringe pump shown in FIG. 1.

FIG. 2C is a top perspective view of an example of a pusher assembly of the carriage shown in FIGS. 2A and 2B.

FIG. 2D is a front view of the pusher assembly of FIG. 2C, showing a front cross-sectional view of one half of the assembly taken along section line A-A shown in FIG. 2C.

FIG. 2E is a back perspective view of the pusher assembly of FIG. 2C shown without an enclosure of the pusher assembly.

FIG. 2F is a side view of an example of a cam mechanism inside the enclosure of the pusher assembly of FIG. 2C.

FIG. 2G is an exploded top perspective view of another example of a pusher assembly for a carriage.

FIG. 2H is a top perspective view of the pusher assembly of FIG. 2G with the enclosure of the pusher assembly removed.

FIGS. 3A to 3C are the top perspective, bottom perspective, and top views, respectively, of an example of a gear train associated with a rack-and-pinion mechanism.

FIGS. 3D and 3E are side cross-sectional views of an example of the rack-and-pinion mechanism with the worm gear engaged and disengaged, respectively.

FIG. 3F is a side view of an example of a rack-and-pinion mechanism with a spring attached to a pivoting mount holding a motor of the drivetrain.

FIG. 3G is a bottom perspective view of another example of a gear train with a worm drive associated with a rack-and-pinion mechanism.

FIG. 3H is an exploded bottom perspective view of the gear train of FIG. 3G

FIG. 3I is a bottom view of the gear train of FIG. 3G with the worm drive engaged.

FIG. 3J is a front cross-sectional view of the gear train taken along section line B-B shown in FIG. 3I.

FIG. 3K is a bottom view of the gear train of FIG. 3G with the worm drive disengaged.

FIG. 3L is a front cross-sectional view of the gear train taken along section line C-C shown in FIG. 3K.

FIG. 3M is a rear cross-sectional view of an alternative example of a gear train.

FIG. 3N is a perspective view of an example of a syringe body grip mechanism.

FIG. 3O is a bottom view of one of two grips of the syringe body grip mechanism of FIG. 3N.

FIG. 3P is a side cross-sectional view of the syringe body grip mechanism of FIG. 3N.

FIG. 4 is a front cross-sectional view of half of the carriage and main body of the syringe pump of FIG. 1.

FIG. 5 is a block diagram of components of the syringe pump of FIG. 1.

FIG. 6 is a schematic of a computer system operable with the syringe pump shown in FIG. 1

FIG. 7A is a perspective view of a syringe pump, with a syringe mounted on the syringe pump.

FIG. 7B is a perspective view of an example of a pusher assembly with a syringe plunger placed on a carriage of the syringe pump of FIG. 7A.

FIG. 8A is a schematic side view of an example of a rack-and-pinion mechanism with a syringe and with a worm gear engaged.

FIG. 8B is a schematic side view of the rack-and-pinion mechanism of FIG. 8A with a syringe and with a worm gear disengaged.

FIG. 9A is a top perspective view of a syringe at least partially mounted in a syringe pump.

FIG. 9B is another top perspective view of a syringe at least partially mounted in a syringe pump.

Like reference symbols in the various drawings indicate like elements.

### DETAILED DESCRIPTION

The present disclosure describes syringe infusion pumps that can accept syringes of a variety of sizes and deliver fluid from the syringe in a precisely controlled manner using a drive mechanism that moves a syringe plunger relative to the body of the syringe. In some implementations, a motor with a worm drive is used to drive a worm gear and gear train, which in turn drives a pinion gear in communication with a rack. In some cases, the gear train and the pinion are included in a drivetrain that could include one or more other components such as one or more drives or other gears. Movement of the rack results in movement of the plunger of the syringe into the syringe body, thus dispensing a medical fluid into a patient's vasculature through a tubing set. The rack can include one or more projections (also referred to as teeth) that engage with a portion of the drivetrain. The rack can be part of the carriage such that the movement of the rack causes movement of the carriage along a longitudinal axis of the carriage relative to a main body of a syringe infusion pump. The carriage can carry the syringe plunger.

The syringe pumps can further include a mechanism (e.g., a clutch mechanism) to disengage the worm drive such that the rack can move independently from the motor. The mechanism to disengage the worm drive can be a mechanical mechanism, an electromechanical mechanism, and/or a combination thereof. Because the rack is movable independently from the motor, the carriage at the rear of the syringe pump can be manually moved into position to install or remove the syringe. The clutch mechanism can be an electromechanical mechanism driven by a secondary motor. In some implementations, the clutch mechanism is a manually actuated mechanism. The clutch mechanism can disengage the worm drive by, for example, moving the worm drive to disengage the worm drive from the gear train. In another example, the clutch mechanism can disengage the worm drive by moving another gear in the gear train to disengage at least a portion of the gear train from the worm drive. In some implementations, a combination of the rack-and-pinion mechanism and the disengageable worm gear can provide an efficient mechanism to deliver small amounts of medical fluid with a high degree of volumetric accuracy. These features can further provide rapid flow initiation and precise flow consistency over long periods of time with improved performance for pressure sensing to detect and respond to tubing occlusion or changes in head height or other environmental issues or fault conditions.

In some implementations, the syringe infusion pumps can reduce siphoning of fluid through medical tubing connected to a syringe mounted in the syringe infusion pump. When a distal end of the medical tubing connected to the syringe is at a lower level than the syringe, the weight of the fluid in the tubing and the negative head height of the tubing can produce a negative pressure within the syringe. The negative pressure can draw a syringe plunger of the syringe into a syringe barrel of the syringe, resulting in unwanted expulsion of fluid through the medical tubing. In some implementations, a syringe infusion pump described herein facilitates mounting of the syringe to the syringe infusion pump such that the syringe barrel cannot move relative to the main body of the syringe infusion pump and the syringe plunger cannot move relative to a movable carriage of the syringe infusion pump. By fixing the position of the carriage relative to the body, the siphoning of the fluid through the medical tubing connected to the syringe may be reduced.

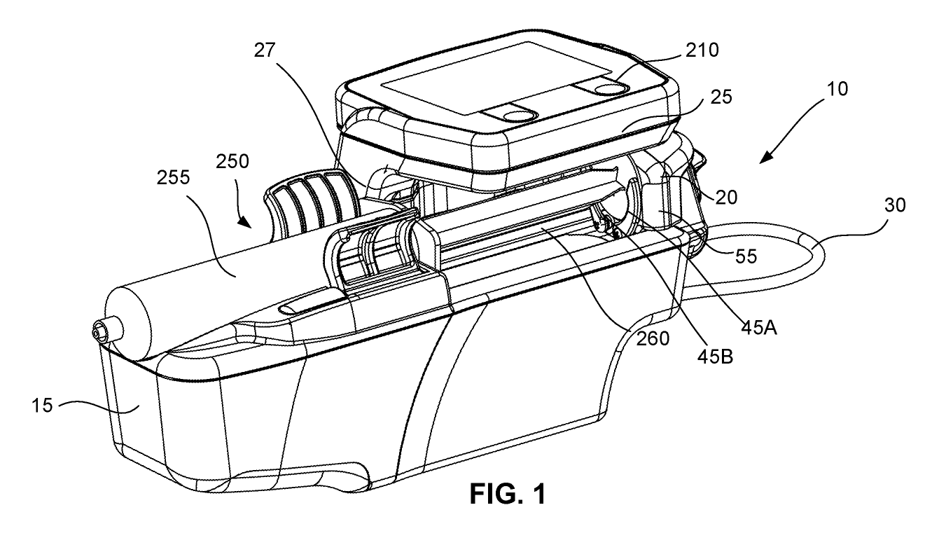

Referring to the example depicted in FIG. 1, a syringe pump 10 includes a main body 15, a carriage 20, and a display 25. As shown, a syringe 250 can be mounted on the syringe pump 10. A loop 30 allows the syringe pump 10 to be hung from a support device such as an IV pole. The main body 15 holds the syringe body 255 and also houses components for electrical and mechanical functionality of the pump 10. The carriage 20 accepts the syringe plunger 260 and moves relative to the main body 15 to produce a pumping action of the syringe 250. When the syringe plunger 260 moves distally (i.e., in a forward direction) relative to the syringe body 255, the volume contained within the syringe body 255 decreases. If the syringe body 255 contains a fluid, the syringe plunger 260 ejects the fluid from the syringe. When the syringe plunger 260 moves proximally (i.e., in a rearward direction) relative to the syringe body 255, the volume contained within the syringe body 255 increases. The carriage 20 moves substantially parallel to a longitudinal axis of the syringe pump 10.

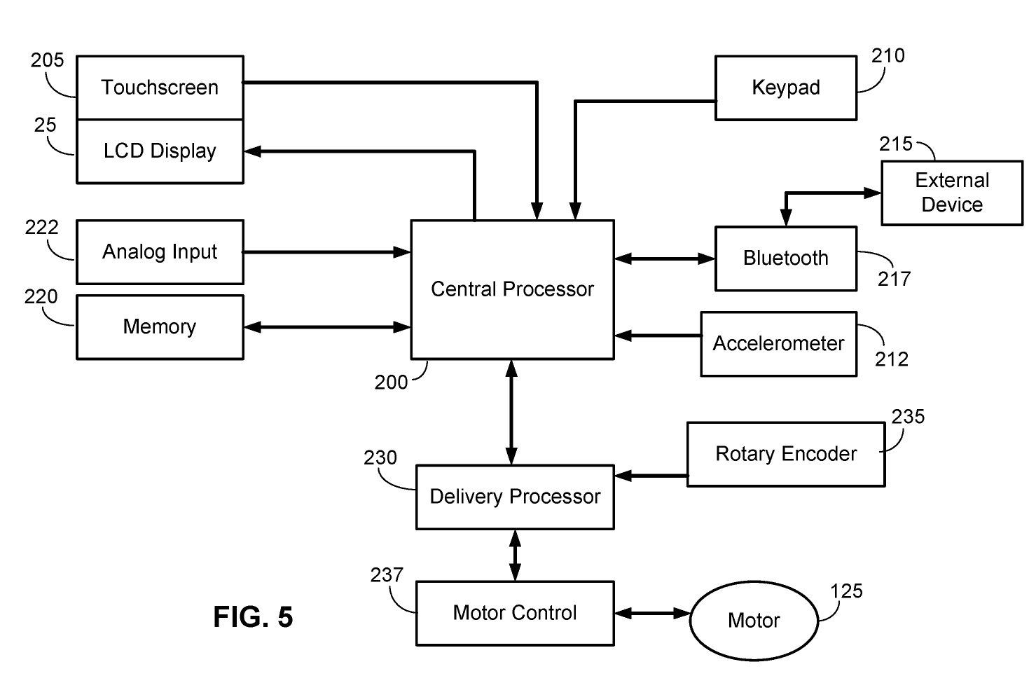

The display 25, operable with a central processor of the syringe pump (described with respect to FIG. 5), serves as a user interface to display information such as fluid delivered, duration of treatment, and other parameters related to the delivery of the medical fluid. A user can also utilize the display 25 as a touchscreen to input information and instructions to the syringe pump 10. The display 25 includes a hinge 27 that allows the display to pivot along an axis parallel to and offset from the centerline of the syringe 250. The display 25 can pivot into a position above the carriage 20 and, in the case when a syringe 250 is deposited into the syringe pump 10, the syringe plunger 260. In some implementations, the hinging action of the display can activate electronic signals that communicate the position of the hinge. The display hinge can also incorporate a friction device in the hinge so that it can be used at various angles to reduce glare and increase ease of use.

### Carriage of Syringe Pump

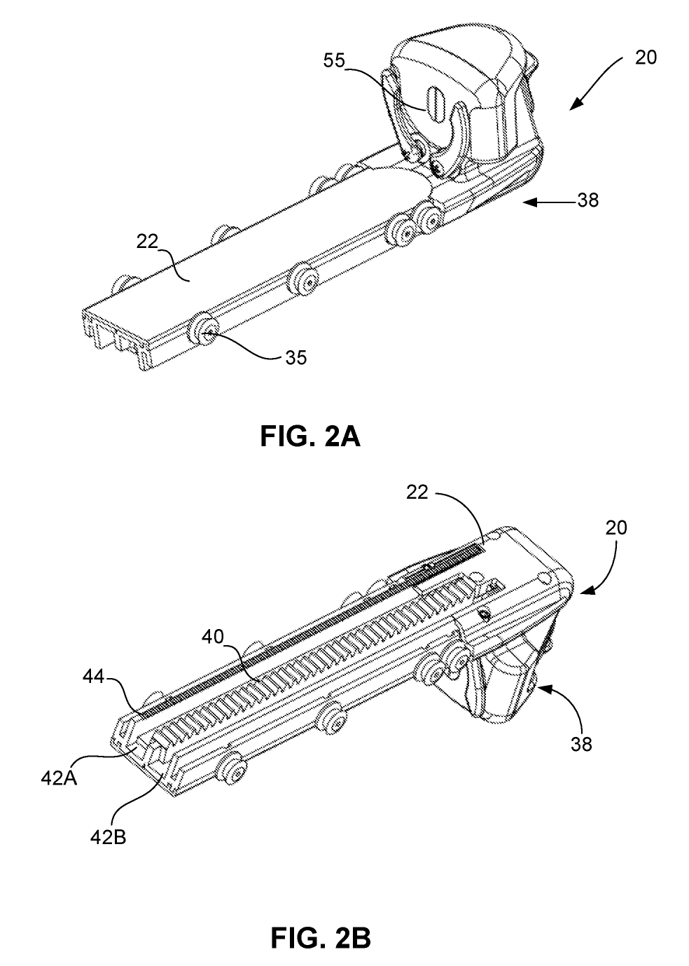

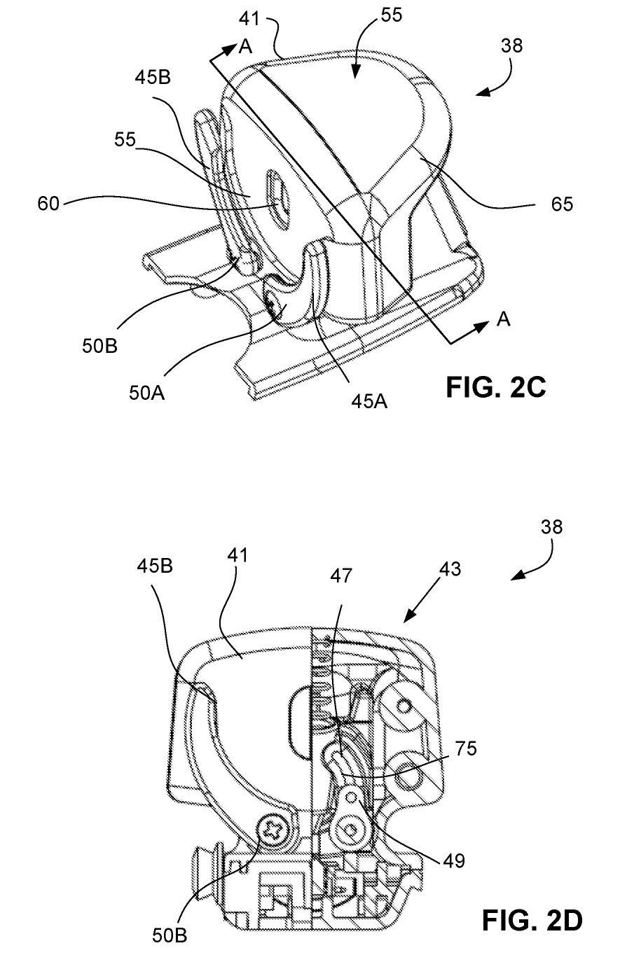

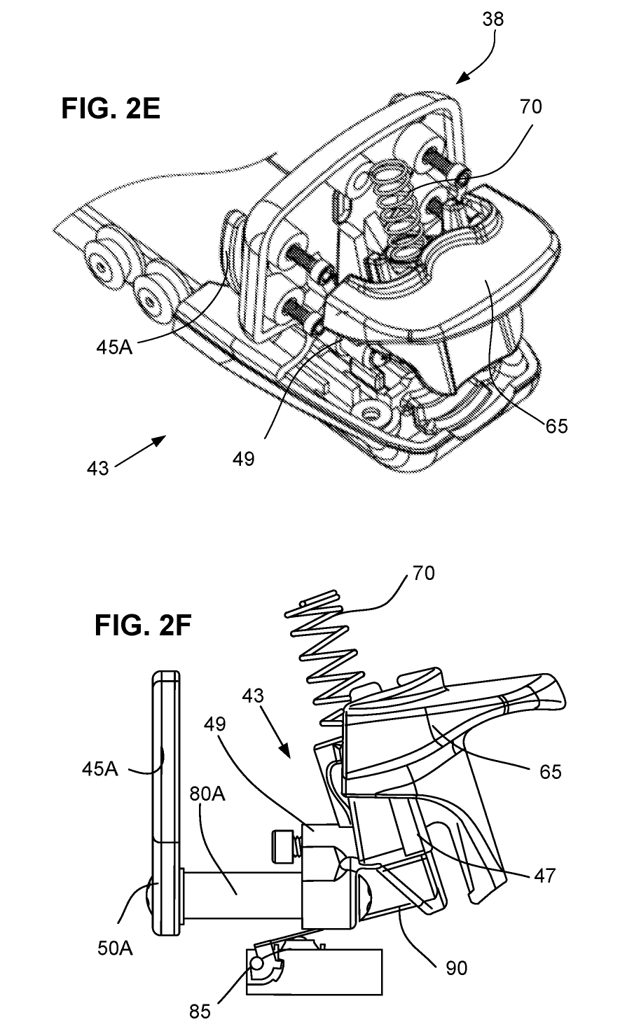

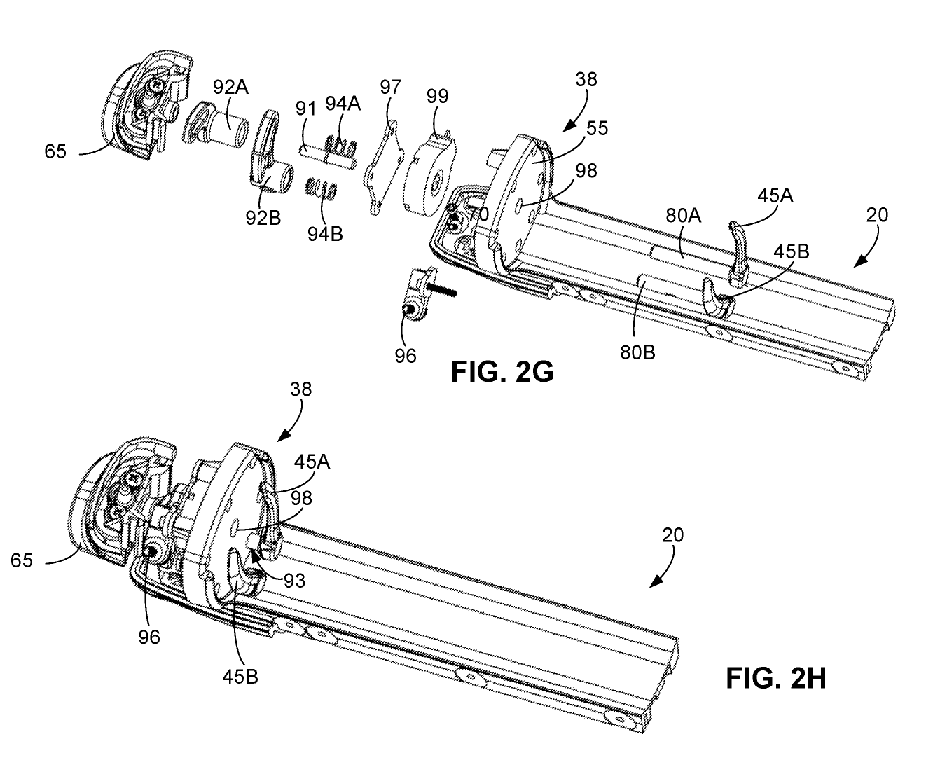

FIGS. 2A to 2E depict the carriage 20, which includes a pusher assembly 38. FIG. 2A is a top perspective view of the carriage 20. FIG. 2B is a bottom perspective view of the carriage 20. Referring to FIGS. 2A and 2B, the carriage 20 has a carriage frame 22 with wheels such as v-profile rollers 35 mounted on both sides, and the pusher assembly 38 in the rear.

The distance between a particular pair of wheels is referred to as a wheelbase. In some implementations, a wheelbase can be substantially equal to one half of a maximum distance traveled by the carriage.

The wheels or v-profile rollers 35 have a rotational symmetry axis passing through the hole that mounts on receiving posts on the sides of the carriage frame 22. The cross-section of a plane encompassing the symmetry axis includes a symmetric profile defined by two V shapes (symmetric about the symmetry axis) with parallel lines connecting the ends of the V shapes. As will be discussed in more detail, one surface formed by the V shape can be steeper than the other surface.

The carriage frame 22 can be made of a rigid material, such as polycarbonate or acrylonitrile butadiene styrene (ABS). The pusher assembly 38 is a rigid structure that pushes the syringe plunger (e.g., the syringe plunger 260 of FIG. 1) during use. The v-profile rollers 35 are mounted within the main body 15 such that they rotate about axes perpendicular to the movement of the carriage 20. To reduce bearing friction, the v-profile rollers can be made of a low-friction material such as nylon or Teflon®.

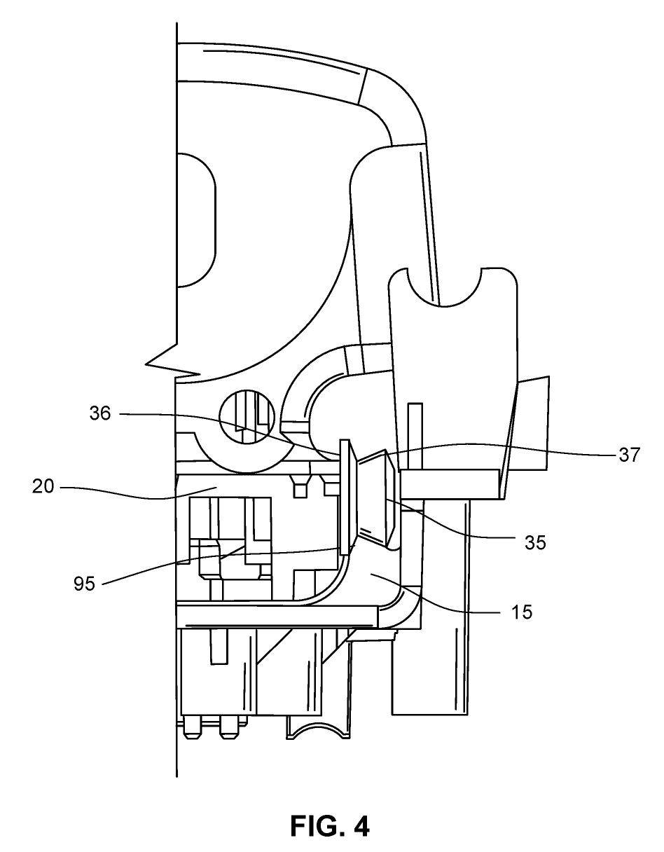

As shown in FIG. 2A and 2B, four v-profile rollers 35 are symmetrically mounted on each side of the carriage frame 22. The v-profile rollers 35 turn freely on axles that project through the carriage frame 22. There can be more or fewer than four such rollers, depending on the size of the overall device. The v-profile rollers 35, as explained in more detail with reference to FIG. 4, guide movement of the carriage 20 relative to the main body of the syringe pump with relatively low friction.

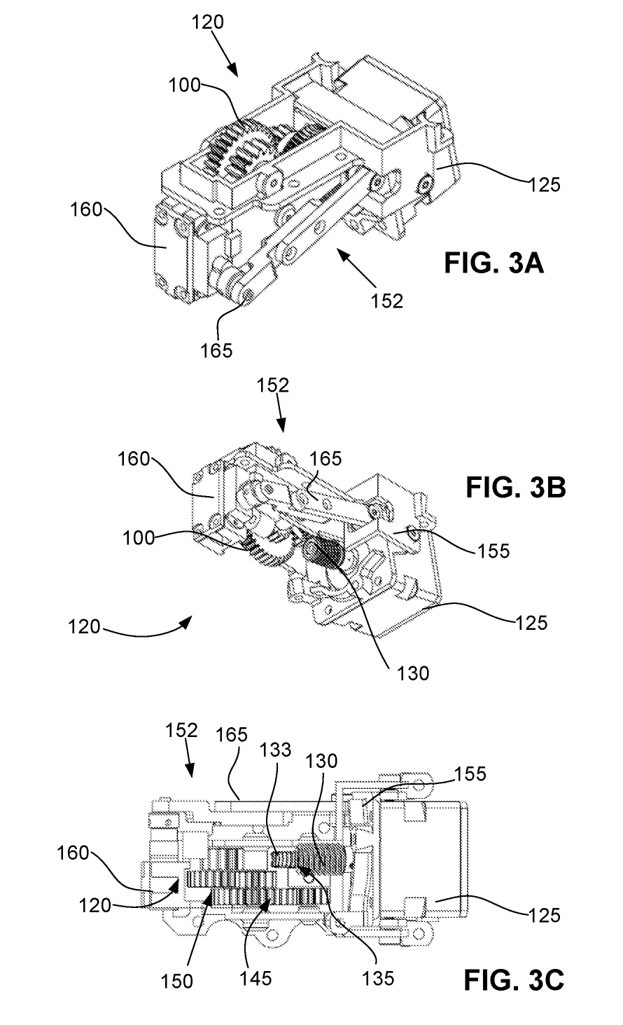

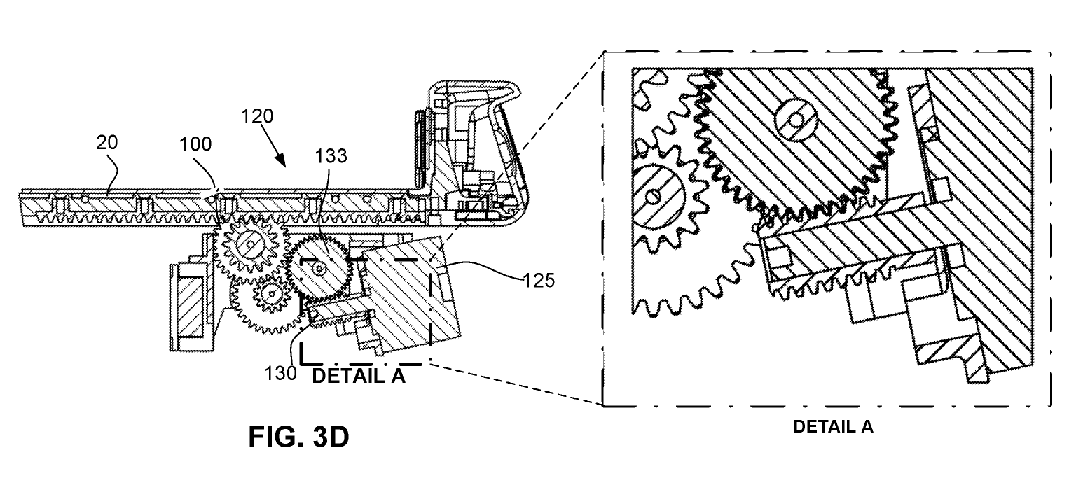

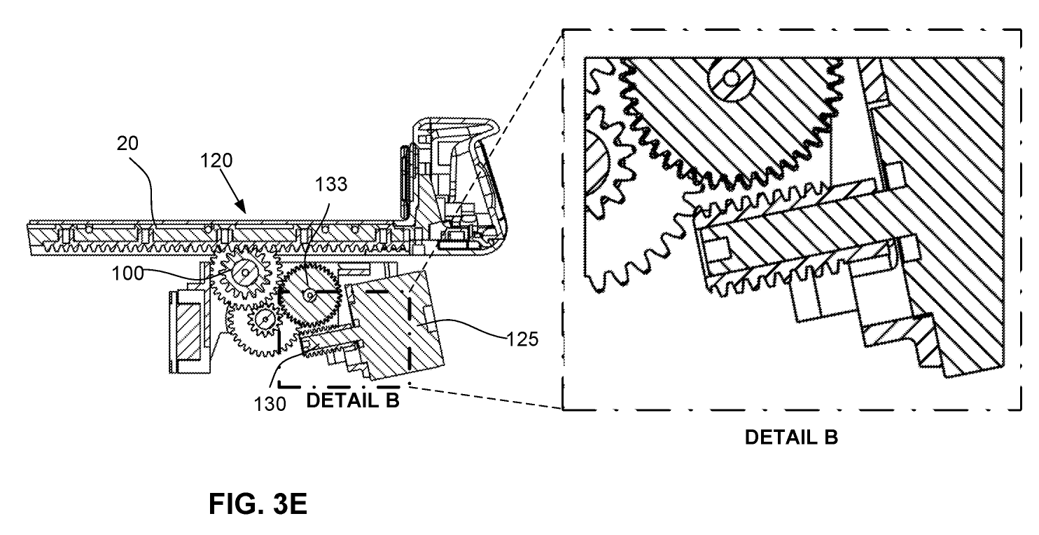

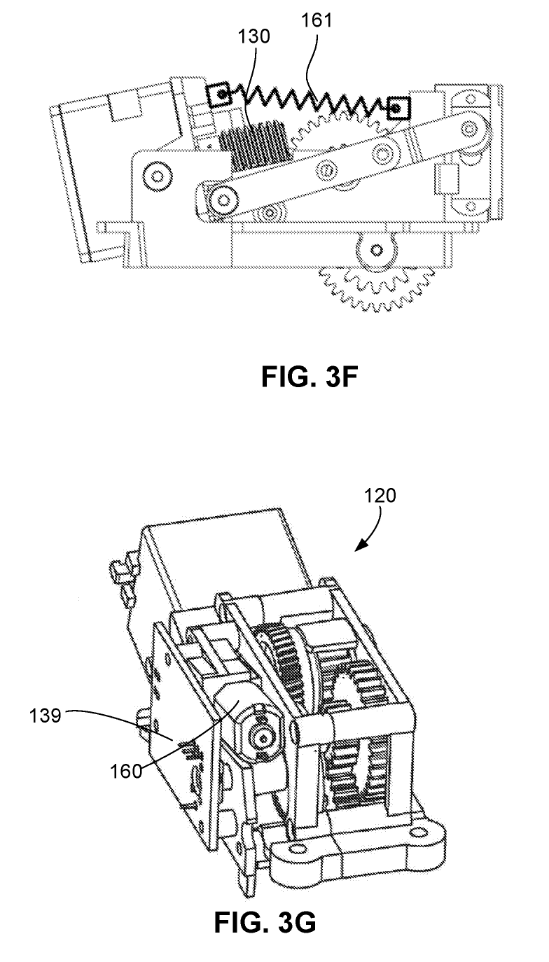

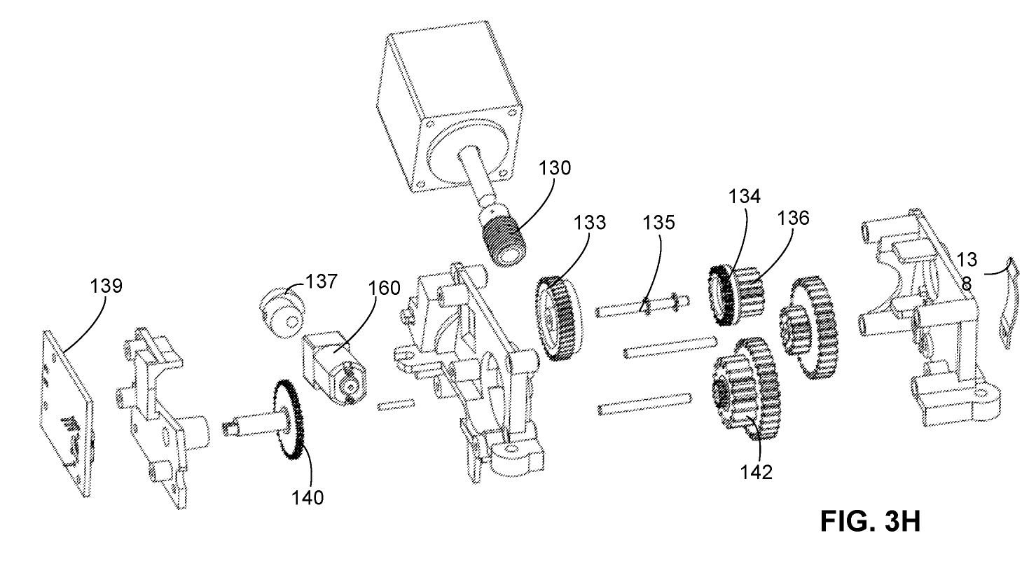

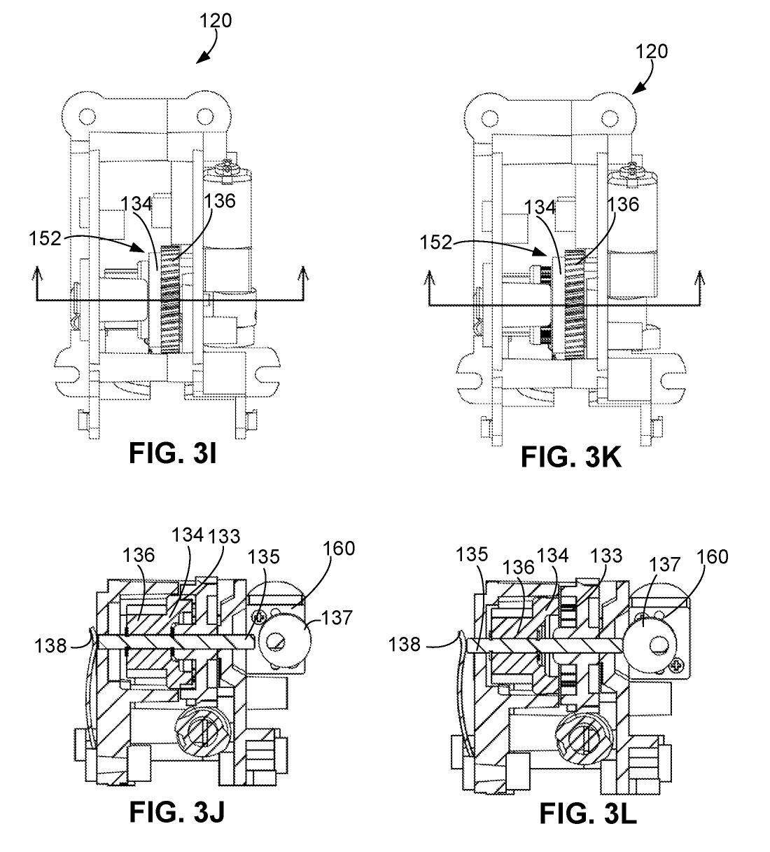

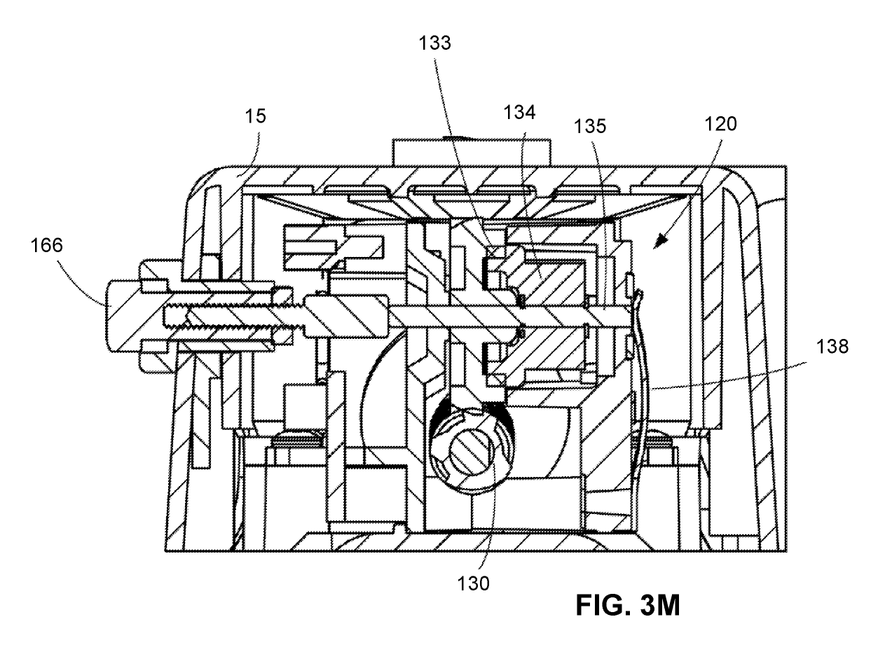

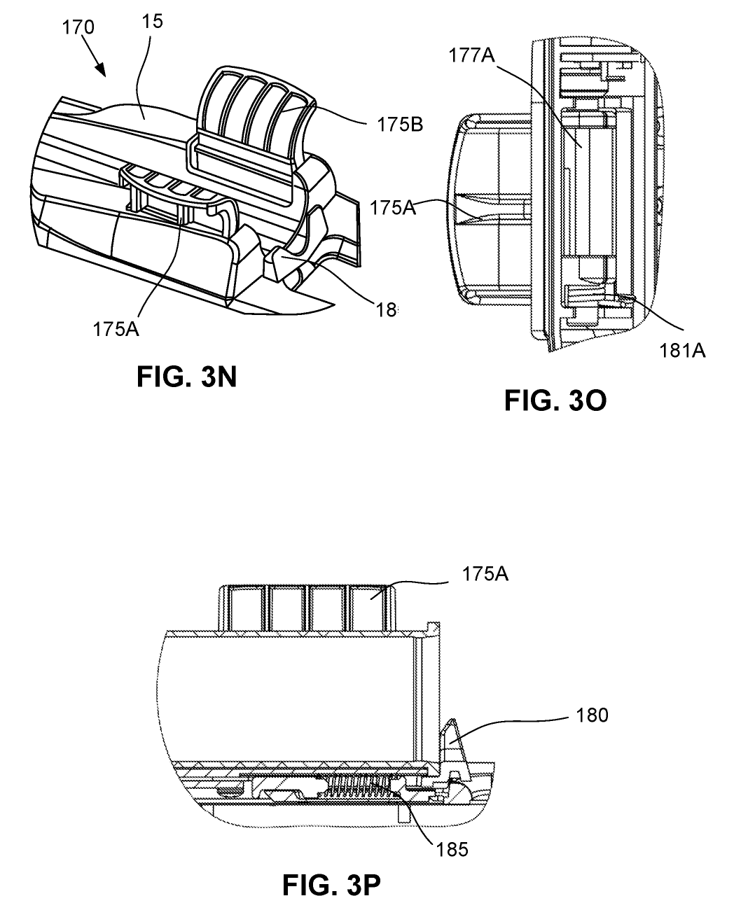

Referring to FIG. 2B, the bottom portion of the carriage 20 further has a gear rack 40 that engages a pinion gear that sits on the main body to form a rack-and-pinion mechanism explained in more detail with reference to FIGS. 3A to 3L. Adjacent to gear rack 40 is a secondary toothed rack 44, suitable for engaging a gear carried on a shaft with an encoder (not shown), such as an absolute encoder positioned on the main body 15, to detect and monitor the position of the carriage and the syringe plunger.

The underside of the carriage frame 22 may further include troughs 42A, 42B integrally molded to the carriage frame 22 to protect electrical and mechanical cables from moving components, such as the v-profile rollers 35 or gear train (not shown). The troughs 42A, 42B travel lengthwise on the lower surface of the carriage frame 22 and can also guide electrical and mechanical cables connecting the pusher assembly 38 to the main body of the device.

FIGS. 2C to 2F show an example of the pusher assembly of the syringe plunger, and FIGS. 2G to 2H depict another example of the pusher assembly of the syringe plunger. With regards to the example of FIGS. 2C to 2F, FIG. 2C shows an isolated view of the pusher assembly 38. FIG. 2D shows a front view of the pusher assembly 38 with a cross-sectional view of half of the assembly to reveal an internal cam mechanism 43 to transmit force on a manual release trigger 65 to operate clamp arms 45A, 45B of the pusher assembly 38. FIG. 2E shows a side/rear view of the pusher assembly 38 without its enclosure 41, revealing the cam mechanism. FIG. 2F shows a side view of the cam mechanism isolated from the pusher assembly 38. Referring to FIG. 2C, the pusher assembly 38 includes the enclosure 41 at the rear of the carriage. The enclosure 41 contains the cam mechanism 43 to operate clamp arms 45A, 45B, which cooperate to center and hold a syringe plunger in place during use.

The clamp arms 45A, 45B are preferably made of a rigid material that allows them to firmly grasp a syringe. For example, the clamp arms can be made of a reinforced polymer such as polycarbonate.

The clamp arms 45A, 45B each rotate about respective clamp arm pivots or hinges 50A, 50B. The user can actuate the manual release trigger 65 to operate the clamp arms 45A, 45B. As the clamp arms 45A, 45B rotate, they engage with circular structural features of the rear portion of syringe plungers, e.g., flanges of syringe plungers. During use, a pusher surface 55 on the pusher assembly 38 can press against the syringe plunger. The pusher surface 55 can contain a force sensor 60 to measure the driving force against the syringe. The pusher surface 55 may also be spring loaded to provide a better grip on the syringe plunger.

Referring to FIGS. 2D to 2F, the cam mechanism 43 is housed inside the enclosure 41. The manual trigger 65 at the rear of the pusher assembly 38 actuates the cam mechanism 43 to release the clamp arms 45A, 45B to load and unload syringes. In the example as shown, the user can actuate the manual trigger 65 to rotate the clamp arms 45A, 45B away from one another. The cam mechanism 43 can include a spring 70 such that the trigger 65 will return to an unactuated state without intervention from the user after the user releases the manual trigger 65. Upon release of the manual trigger 65, the spring 70 causes the clamp arms 45A, 45B to rotate toward one another and can engage with a syringe plunger.

FIGS. 2D to 2F show the cam mechanism 43 of the clamp arm 45A exposed on one side of the pusher assembly 38. The same mechanism can be used for the clamp arm 45B as well. The cam mechanism 43 includes a sliding cam component 47 and a cam follower arm 49. A fastener can couple the trigger 65 to the sliding cam component 47 via, and a shaft 80A can couple the cam follower arm 49 to the clamp arm 45A. The sliding cam component 47 has a track 75 that accepts the cam follower arm 49, which is coupled to the clamp arm 45A. As a result, as the trigger 65 is actuated, the cam follower arm 49 follows the track 75 of the sliding cam component 47, resulting in a rotation of the cam follower arm 49. Since the cam follower arm 49 are coupled, the clamp arm 45A rotates about the shaft 80A.

During actuation of the cam mechanism 43, the sliding cam component 47 can also push against an electrical switch 85 to release the drive mechanism to allow free manual positioning of the carriage. For example, the sliding cam component 47 can push against a pivot arm 90 that presses the electrical switch 85. The pivot arm 90 can be biased by a torsional spring such that the electrical switch 85 is released when the manual trigger 65 is released.

In the example shown in FIGS. 2D to 2F, the cam follower arm 49 and the trigger 65 are separable components coupled with a fastener. In other implementations, the trigger 65 and the sliding cam component can be integrally molded.

While the trigger 65 is described as a single trigger to operate both the electrical switch and the cam mechanism, the trigger 65 could be separable into two different buttons. For example, one button or trigger 65 could toggle the electrical switch, and the other button could toggle the cam mechanism.

In the implementation shown in FIG. 2E, the trigger 65 mechanically actuates the cam mechanism. In other implementations, the trigger 65 could be an electrical switch that actuates a motor driving the cam mechanism.

While both clamp arms are described to have their respective portions of the cam mechanism to drive their rotation, in some implementations, the trigger 65 actuates only one of the clamp arms. In some cases, one or both clamp arms can be spring-loaded, and the syringe can be positioned with only one clamp arm released.

While the frame, gear rack, wheel mount, troughs, and base for the pusher assembly have been described as separate components, in some implementations, these components can be elements of a single, monolithic component. For example, the single component could be made from a reinforced polymer, e.g., glass or carbon fiber-reinforced polycarbonate or ABS, using processes such as injection molding or 3D printing. The single component could further include additional cover sections to provide protection for internal components, additional stiffness, and a cosmetic appearance.

In some implementations, the carriage may further include a sensor, which upon placement of the syringe plunger flange, allows a determination if the syringe plunger flange is securely seated between the rotating clamp arms and the pusher surface. The sensor can transmit a signal to the central processor of the syringe pump to indicate that the syringe plunger flange is securely seated or is not securely seated. If the flange is not securely seated, the central processor can issue an alarm to inform the user that the flange requires attention.

In some implementations, the pusher assembly can include a force sensor 60 (shown in FIG. 2C) that measures the force with which the pusher assembly drives the syringe plunger. The force sensor can be configured to provide a feedback signal to one or more processing devices (e.g., the central processor of the syringe pump) that control operation of the motor such that the thrust exerted by the motor on the plunger can be controlled based on the feedback. The force sensor can transmit the signal generated in response to measuring the force to the central processor of the syringe pump. The central processor of the syringe pump can then determine if the driving force has exceeded a predetermined limit. For example, if the force with which the pusher assembly drives the syringe plunger exceeds a predetermined limit or falls outside of a particular interval, the central processor can issue an alert to the user to the source of the excessive force. The excessive force may be caused by, for example, an occlusion in the fluid outflow tubing or a high flow rate into a narrow vascular access device. In response to the excessive force, the central processor can additionally or alternatively control the speed and/or direction of the motor by providing an appropriate control signal to the motor.

In addition to being able to rotate about the pivots 50A, 50B, in some implementations, the clamp arms 45A, 45B also translate along the longitudinal axis of the pusher assembly 38. By being movable in a forward direction along the longitudinal axis of the pusher assembly 38 relative to the pusher surface 55, the clamp arms 45A, 45B may accommodate larger and thicker plunger flanges. Greater volume syringes, such as, for example, 60 cubic centimeter to 120 cubic centimeter syringes, can have wider or thicker plunger flanges. By being movable relative to the pusher surface 55, the clamp arms 45A, 45B may capture a variety of syringes having varying widths and thicknesses.

The clamp arms 45A, 45B can also be configured to exert a force in a rearward direction on a plunger flange of a syringe loaded into the clamp arms 45A, 45B. A spring mechanism can bias the clamp arms 45A, 45B to press against the plunger flange loaded into the clamp arms 45A, 45B. Because the clamp arms 45A, 45B are movable relative to the pusher surface 55, the syringe plunger when loaded into the pusher assembly 38 can be movable relative to the pusher surface 55 as well. In some examples, fluid pressure within the syringe may directly exert a force on the syringe plunger such that the syringe plunger and the clamp arms 45A, 45B may move relative to other portions of the pusher assembly 38. A force from the clamp arms 45A, 45B pulling the syringe plunger toward the pusher surface 55 increases an amount of force in the forward direction on the plunger required to move the plunger relative to other portions of the pusher assembly 38. This movement of the plunger relative to the pusher assembly 38 may cause fluid to be ejected from the syringe. The force from the clamp arms 45A, 45B pulling the plunger toward the pusher surface 55 may reduce the risk of accidental fluid delivery due to application of inadvertent forces to the syringe plunger. For example, the increased force requirement may reduce the risk of siphoning. The force of the clamp arms 45A, 45B against the plunger flange may reduce the amount of play or movement of the plunger flange relative to the clamp arms 45A, 45B after the plunger has been loaded into the pusher assembly 38.

FIGS. 2G and 2H show an example of the pusher assembly 38 whose clamp arms 45A, 45B can translate along the longitudinal axis of the carriage 20. FIG. 2G shows an exploded view of the pusher assembly 38, and FIG. 2H shows the pusher assembly 38 with the enclosure removed to reveal an internal mechanism to transmit force applied on the trigger 65 to the clamp arms 45A, 45B.

In the pusher assembly 38 of FIGS. 2G and 2H, a spring (not shown) biases the trigger 65 of the pusher assembly 38 such that the trigger 65 returns to an unactuated position. In contrast to the pusher assembly 38 of FIGS. 2D to 2F, the trigger 65 of FIGS. 2G to 2I moves upward toward an actuated position. The spring biasing the trigger 65 of FIGS. 2G to 2H can bias the trigger 65 downward to return the trigger 65 to the unactuated position. The trigger 65 can nest into two guided slots (not shown) that guides the motion of the trigger 65 during actuation. The trigger 65 moves in an upward direction angled slightly toward the forward direction during actuation, and the spring downwardly biases the trigger 65 back toward the unactuated position.

In some implementations, a pin 91, which is attached to the trigger 65, bisects two rotating guides 92A, 92B fixed to the clamp arms 45A, 45B, respectively. The pin 91 and the rotating guides 92A, 92B transfer the motion of the trigger 65 into a symmetric or substantially symmetric rotation of the clamp arms 45A, 45B. When the trigger 65 is actuated, the motion of the pin 91 caused by the movement of the trigger 65, results in rotation of the guides 92A, 92B. The guides 92A, 92B in turn cause the clamp arms 45A, 45B to rotate.

Because the path of the trigger 65 is slightly angled in the forward direction, the pin 91 exerts a forward force on the rotating guides 92A, 92B. Due to the forward force from the pin 91, the rotating guides 92A, 92B translate in the forward direction, as well as rotate, when the trigger 65 is actuated. The clamp arms 45A, 45B translate in the forward direction with the guides 92A, 92B such that the clamp arms 45A, 45B move away from the pusher surface 55 of the pusher assembly 38. When the trigger 65 is actuated, a gap 93 forms between the clamp arms 45A, 45B and the pusher surface 55. The gap 93 accommodates the plunger flange and allows for the plunger flange to be easily mounted into the clamp arms 45A, 45B. In some implementations, the gap 93 is between 0.635 millimeters and 1.02 millimeters (0.025 inches and 0.040 inches). The rotation and translation of the clamp arms 45A, 45B, upon actuation of the trigger 65, enable the pusher assembly 38 to accommodate syringe plunger flanges of varying diameters and thicknesses.

The clamp arms 45A, 45B can be spring-loaded so that the clamp arms 45A, 45B are biased toward the pusher surface 55. For example, the pusher assembly 38 can include retraction springs 94A, 94B, each of which has one end grounded within the enclosure 41 of the pusher assembly 38. The other end of the retraction springs 94A, 94B can bear against one of the rotating guides 92A, 92B, respectively, or one of the clamp arms 45A, 45B, respectively. The shafts 80A, 80B of the clamp arms 45A, 45B can rotate within bosses or bearing holes within the enclosure 41, and the retraction springs 94A, 94B can be grounded against those bearing holes. The retraction springs 94A, 94B are loaded such that they bias the clamp arms 45A, 45B toward the pusher surface 55. With this type of loading, after the trigger 65 is moved to an actuated position, and the clamp arms 45A, 45B translate away from the pusher surface 55, the retraction springs 94A, 94B are in tension and tend to pull the clamp arms 45A, 45B back toward the pusher surface 55. After the trigger 65 is released, the tension force from the retraction springs 94A, 94B retract the clamp arms 45A, 45B back toward the pusher surface 55.

The shafts 80A, 80B are positioned within the bearing holes such that the forward movements of the shafts 80A, 80B are limited even when the trigger 65 is not actuated. A forwardly directed force on the clamp arms 45A, 45B causes the clamp arms 45A, 45B to translate in the forward direction. The amount of translation can be limited by, for example, the rotating guides 92A, 92B contacting internal surfaces of the enclosure 41 after the clamp arms 45A, 45B translate a certain distance. In some cases, the shafts 80A, 80B may each include a protrusion that contacts an internal surface of the enclosure 41 or a surface of the bearing holes to limit the distance travelled by the clamp arms 45A, 45B.

Similar to the pusher assembly 38 depicted in FIGS. 2A to 2F, the pusher assembly 38 of FIGS. 2G to 2H can also include electromechanical components for controlling sensing and driving functions of the syringe pump. As described with respect to FIG. 2C, the pusher assembly 38 can include the force sensor 60 to determine the amount of force applied to a syringe plunger loaded into the pusher assembly 38. The pusher assembly 38 can additionally include a retaining plate 97 to hold a load cell or force sensor 99 in place against a main body of the pusher assembly 38.

Further, as described with respect to FIG. 2D to 2F, the electrical switch 85, upon actuation, generates an electrical signal to cause the release of the drive mechanism to enable free manual positioning of the carriage of the syringe pump. The pusher assembly 38 in FIGS. 2G to 2H can include similar features. An electrical switch mounted on a bracket 96 positioned within the enclosure of the pusher assembly 38 can be triggered by rotation of one of the rotating guides 92A, 92B (e.g., the rotating guide 92B as shown in FIG. 2G).

The pusher assembly 38 can include a mechanism to detect proper placement of the syringe flange within the pusher assembly 38. The mechanism can detect whether the syringe flange sits substantially flush with the pusher surface 55. For example, the pusher assembly 38 can include a thrust pin positioned along a hole 98 centrally located on the pusher surface 55. The thrust pin can cause motion of a clevis arm that, in turn, triggers a micro-switch within the pusher assembly 38. The micro-switch can cause an electrical signal to be sent to the central processor of the syringe pump that indicates that a syringe plunger flange has been properly loaded into the pusher assembly 38.

### Main Body of Syringe Pump

FIGS. 3A to 3E show aspects of the main body 15 of the syringe pump. FIGS. 3A to 3C depict the drivetrain 120 that induces the linear motion of the carriage through the rack-and-pinion mechanism. FIG. 3A shows a top/side view of the drivetrain 120 in the main body 15. FIG. 3B shows a bottom/side view of the same drivetrain 120. The drivetrain 120 drives the pinion gear 100 that induces the linear motion of the carriage via the rack on the underside of the carriage. Referring to FIG. 3B, a motor 125 goes through a three stage gear reduction 134, 145, and 150 used to reduce the speed and increase the torque of the motor 125, which can be, for example, a hybrid type stepper motor. The first stage 134 includes a worm drive 130, and the worm drive 130 is directly coupled to the motor shaft. The portion of the shaft that engages with the worm gear may be referred to as a worm drive. The second stage 145 and third stage 150 include spur gear reductions. For example, when a first spur gear meshes with a second spur gear in the drivetrain 120, the first spur gear may be larger than the second spur gear such as to produce a gear reduction (i.e., the tangential velocity on the circumference of the first gear is faster than the tangential velocity on the circumference of the second gear).

The drivetrain 120, with the worm drive 130, can convert low torque and high speed rotation of a drive motor, such as the motor 125, to a high force and low speed linear motion of the carriage relative to the main body 15. The worm drive 130, when engaged with the drivetrain 120, may limit an amount of backdriving of the drivetrain. When the worm drive 130 is engaged to the worm gear 133, the worm drive 130 may prevent the motor 125 from being backdriven. As a result of this engagement between the worm drive 130 and a portion of the drivetrain 120 engaged with the rack of the carriage, the main body and carriage cannot be moved relative to one another whenever the motor 125 is not turning. When the worm drive 130 is engaged, the worm drive 130 can limit unintentional movement of the carriage relative to the main body 15 because the worm drive 130 cannot be easily backdriven.

The drivetrain 120 can include a clutch mechanism 152 that can disengage the entire drivetrain 120 or a portion of the drivetrain 120 from the worm drive 130. The clutch mechanism 152 decouples the carriage from the portion of the drivetrain 120 engaged to the worm drive 130. After the decoupling occurs, the carriage and the portion of the drivetrain 120 can be driven both forwards and backwards. As a result, the carriage can be moved, e.g., along the longitudinal axis of the carriage, with low impediment for syringe loading. The clutch mechanism 152 facilitates decoupling. For example, when the clutch mechanism 152 is activated to disengage at least a portion of the drivetrain 120 from the worm drive 130, the user can manually re-position the carriage of the syringe. After the syringe is loaded into the carriage, the clutch mechanism 152 can be deactivated. Upon deactivation, the carriage and the drivetrain 120 can be re-engaged with the worm drive 130, and, in some cases, a spring can bias the worm drive 130 to re-engage with the drivetrain 120 and the carriage.

By limiting the amount of backdriving that can occur when engaged with the carriage through the drivetrain 120, the worm drive 130 can limits inadvertent withdrawal of fluid that can occur during operation of the syringe pump. For example, when a fluid-filled syringe is already loaded onto the syringe pump, the user may inadvertently apply a rearward force on the carriage during an operation of the syringe pump. The worm drive 130, by causing an impediment to being backdriven, can substantially prevent that force from causing movement of the syringe plunger relative to the syringe body, thereby reducing undesired withdrawal of fluid into the syringe.

Referring to FIGS. 3A to 3C, the clutch mechanism 152 engages or disengages the worm drive 130 from the worm gear 133 such that the entire drivetrain 120 is disengaged from the worm drive 130. Referring to FIG. 3D, when the worm drive 130 is engaged to the worm gear 133, the worm drive 130 prevents the motor 125 from being backdriven so the main body and carriage are locked together when the motor 125 is not turning. As shown in detail A of FIG. 3D, when the worm drive 130 is engaged to the worm gear 133, the thread of the worm drive 130 engages with the teeth of the worm gear 133.

Referring to FIG. 3E, when the worm drive 130 is disengaged, the user can manually reposition the carriage relative to the main body. As shown in detail B of FIG. 3E, when the worm drive 130 is disengaged from the worm gear 133, the thread of the worm drive 130 does not contact the teeth of the worm gear 133. Referring back to FIGS. 3A to 3C, the motor 125 is further fixed to a pivoting mount 155. Rotating the mount 155 over a small angle causes the worm drive 130 to move out of mesh with the worm gear 133, allowing the spur gears in the gear train, the pinion gear 100, and the rack to move freely. Referring briefly to FIG. 3F, a spring 161 holds the worm drive 130 in the meshed position, with a mechanical stop acting to keep the worm drive 130 and the worm gear 133 (not visible in FIG. 3F) at the correct mesh distance. When the clutch mechanism is deactivated (e.g., the trigger is released), the worm drive 130 re-engages with the worm gear 133.

Referring back to FIGS. 3A to 3C, a secondary motor 160 and linkage mechanism 165 operates against the spring 161 to pull the motor 125 and worm drive 130 out of mesh with the worm gear 133. The secondary motor 160 is activated by the electrical switch described earlier, which the user actuates by depressing the manual trigger 65 described above. The manual trigger 65 thus activates the clutch mechanism such that the clutch mechanism disengages the worm drive 130 from the spur gears of the gear train. The secondary motor 160 can backdrive if power is removed so as to engage the worm drive 130 to the worm gear 133 in the event of a power failure. As a result, the worm drive 130 moves into mesh with the worm gear 133, preventing the carriage from being easily back-driven thereby reducing accidental carriage movement.

While the drivetrain is described to include a three-stage speed reduction gear train, the number of stages and amount of speed reduction can vary depending on the implementation. For example, for high-volume syringes, it may be beneficial to have a lower speed reduction to deliver medical fluid faster.

While a linkage mechanism is described to keep the worm drive in mesh with the adjacent worm gear, other implementations can incorporate a different mechanism. For example, another gear train or a cable-driven mechanism could drive the spring to disengage the worm drive from the worm gear. An additional gear train can include a combination of spur gears and bevel gears to achieve a desired speed and directionality.

In some implementations, the pinion gear can be an anti-backlash gear to improve precision of motion transfer from the pinion gear to the gear rack and to reduce the effects of gear wearing. While the pinion gear is shown and described to be positioned to mesh with the gear rack, in some implementations, a feature can be added so as to have a force act on the pinion gear to keep tighter engagement with the gear rack. For example, the pinion gear can be spring-loaded. The additional spring can be attached to the pinion gear so that the force of spring keeps the pinion gear in tighter mesh with the gear rack, thus reducing backlash.

While the drive motor 125 has been described as a hybrid stepper analog motor, in some implementations, the drive motor 125 can be stepper motor or an analog motor. While the secondary motor 160 is shown and described to pivot the motor 125 and worm drive 130 out of mesh with the worm gear 133, a non-motorized mechanism could be used as well. In some implementations, the secondary motor 160 can be an analog motor, a stepper motor, or a hybrid stepper analog motor.

The worm drive 130 and motor pivoting mount 155 can include an additional trigger that the user can depress to manually rotate the pivoting mount 155 to disengage the worm drive 130 from the worm gear 133. In the implementation as shown in FIGS. 3A to 3F, the pivoting mount 155 allows the motor to be rotated away from the worm gear 133. In other implementations, instead of pivoting, the mount 155 could translate in a direction that disengages the worm drive 130 from the worm gear 133.

In some implementations, rather than translating the worm drive 130 away from the worm gear 133 to disengage the worm drive 130 from the worm gear 133, the clutch mechanism 152 can cause another gear of the drivetrain 120 to translate away from the worm gear 133. The translation of this gear away from the worm gear 133 can disengage that gear from the worm gear 133 to decouple a portion of the drivetrain 120 from the worm drive 130 and the worm gear 133. The clutch mechanisms depicted in FIGS. 3G to 3L and FIGS. 3A to 3F differ in some regards, such as, for example, the mode of movement of drive components to cause disengagement of the worm drive 130 from the drivetrain 120. In particular, instead of rotating the worm drive 130 to disengage the worm drive 130 from the drivetrain 120, the clutch mechanism of FIGS. 3G to 3L translates a clutch gear 134 on a shaft 135 away from the worm gear 133 to disengage the clutch gear 134 from the worm gear 133.

FIG. 3G shows a bottom perspective view of the example gear box, and FIG. 3H shows an exploded view of the gear box. The clutch gear 134, by disengaging from the worm gear 133, causes the drivetrain 120 to be divided into several separated portions disengaged from one another. These portions can include (i) the worm gear 133 engaged to the worm drive 130, (ii) the clutch gear 134, and (iii) the remainder of the drivetrain 120 engaged to the gear rack of the carriage. The gears in the portion of the drivetrain 120 engaged with the gear rack can rotate in a manner independent from the rotation of the worm drive 130, as that portion is disengaged from the worm drive 130. Thus, the carriage with the gear rack can move with low impediment when the clutch mechanism 152 is activated to disengage the worm drive 130. When the portions are engaged, the worm drive 130 drives the worm gear 133. The worm gear 133 engages with the clutch gear 134, which rotates with the spur gear 136. The spur gear 136 drives gears 141 which in turn drives gears 142. The gears 142 can include a pinion gear of the rack-and-pinion mechanism of the carriage. Rotation of the gear 142 therefore can cause linear movement of the carriage.

The clutch mechanism 152 can include a secondary motor 160 that causes the clutch gear 134 to move away from the worm gear 133. The secondary motor 160 rotates a cam 137 that pushes the shaft 135 away from the secondary motor 160. Moving with the shaft 135, the clutch gear 134 thus moves away from the worm gear 133. Actuating the trigger 65 can cause the clutch mechanism 152 to activate, thereby causing disengagement of the clutch gear 134 from the worm gear 133. The clutch gear 134 can also be biased by a spring 138 toward the cam 137. As a result, in response to the clutch gear 134 moving away from the worm gear 133, the spring 138 generates a force to push the clutch gear 134 back towards the worm gear 133. When the clutch mechanism 152 is deactivated, the spring 138 pushes the clutch gear 134 back into engagement with the worm gear 133.

FIGS. 3I and 3J shows a bottom view and a front cross-sectional view, respectively, of the drivetrain 120 when the clutch gear 134 is engaged with the worm gear 133. The outer teeth of the worm gear 133 is engaged to the worm drive 130, and in the implementations described with respect to FIGS. 3G to 3L, the worm gear 133 is engaged to the worm drive 130 during the operation of the syringe pump.

When the clutch gear 134 is engaged with the worm gear 133, the teeth of the clutch gear 134 engage inner teeth of the worm gear 133. The worm gear 133 and the clutch gear 134 form a mechanism similar to a dog clutch. The clutch gear 134 is a male clutch gear whose outer teeth engage the inner teeth of the worm gear 133 serving as the female clutch gear.

The clutch gear 134 and a spur gear 136 are both positioned on the shaft 135. Rotation of the clutch gear 134 causes rotation of the shaft 135 and thus the spur gear 136. The spur gear 136 in turn drives the remaining portion of the drivetrain 120 engaged with the rack of the carriage to cause linear motion of the carriage. When the clutch gear 134 is engaged as shown in FIGS. 3I and 3J, the gear rack of the carriage is engaged with the worm drive 130. In such a configuration, a rearward motion of the carriage is limited due to the resistance or impediment provided by the worm drive 130.

FIG. 3K shows the bottom view of the drivetrain 120 (as shown in FIG. 3I) but with the clutch gear 134 disengaged from the worm gear 133. FIG. 3L shows the cross-sectional view depicted in FIG. 3K with the clutch gear 134 disengaged. As FIGS. 3I to 3L depict, when the clutch gear 134 is engaged to the worm gear 133 (FIGS. 3I and 3J), the clutch mechanism 152 can be activated to cause the clutch gear 134 to move away from the worm gear 133 to disengage from the worm gear 133 (FIGS. 3K and 3L). When the clutch gear 134 is disengaged from the worm gear 133 (FIGS. 3K and 3L), rotation of the spur gear 136 can occur in both directions. This rotation is independent of the motion of the worm drive 130 because the spur gear 136 is not engaged with the worm drive 130 (FIGS. 3K and 3L) through the clutch gear 134 and the worm gear 133. As a result, the carriage engaged with the spur gear 136 can be manually moved in both directions along the longitudinal axis of the carriage.

To re-engage the clutch gear 134 with the worm gear 133, the clutch gear 134 is moved toward the worm gear 133 such that the outer teeth of the clutch gear 134 are engaged with the inner teeth of the worm gear 133 (FIGS. 3I and 3J). The spring 138 can cause the clutch gear 134 to move back into engagement with the worm gear 133 after the clutch mechanism is deactivated.

To enable the engagement between the outer teeth and the inner teeth, the clutch gear 134 may rotate slightly so that the outer teeth of the clutch gear 134 and the inner teeth of the worm gear 133 are aligned. The slight rotation of the clutch gear 134 may result in rotation of the spur gear 136, which can, in turn, drive the remainder of the drivetrain 120. Similarly, in the implementations described with respect to FIGS. 3A to 3F, the worm gear 133, when re-engaged to the worm drive 130, may rotate by a small amount such that the pitch of the worm drive 130 engages with the teeth of worm gear 133. The small rotation of the worm drive 130 may cause the remainder of the drivetrain 120 to be driven by a corresponding small amount.

In some implementations, the position of the clutch mechanism 152 may reduce both an inadvertent movement of the carriage, and wear of the clutch mechanism 152 during re-engagement of the clutch mechanism 152. Because the clutch mechanisms 152 described with reference to FIGS. 3A to 3L disengage a portion of the drivetrain 120 near the motor 125, any rotation of the worm gear 133 or the clutch gear 134 during alignment can undergo gear reduction through the remainder of the drivetrain 120. The small rotation caused by the re-engagement thus results in a proportionally smaller linear movement of the carriage because the small rotation is further reduced through the gear reduction of the remainder of the drivetrain 120. The resulting small movement of the carriage during re-engagement of the drivetrain 120 reduces the possibility and/or amount of fluid being inadvertently expelled from the syringe or drawn into the syringe.

In addition, torque loads on the gears of the drivetrain 120 are lowest near the motor end of the drivetrain 120. In this regard, contact stresses and shear loads between the clutch gear 134 and the worm gear 133 are lower than if the clutch gear 134 were positioned within the drivetrain 120 closer to the carriage. The reduced contact stresses and shear loads reduce wear on the gears.

Referring back to FIGS. 3G and 3H, in some implementations, the gear box includes a circuit board 139 that is operable with the central processor of the syringe pump to provide feedback signals to the central processor and receive control signals from the central processor. The circuit board 139 can be connected to a position-sensing gear 140 that senses a position of the carriage. In some implementations, the position-sensing gear 140 engages the secondary tooth rack 44 as described with respect to FIG. 2B. As the position-sensing gear 140 rotates, the central processor can receive signals from an encoder connected to a shaft carrying the position-sensing gear 140 to determine a position of the carriage 20 relative to the main body of the syringe pump.

While the clutch mechanism 152 has been described as an electromechanical mechanism, in some implementations, the clutch mechanism can be manually activated or deactivated. The clutch mechanism can be activated through a separate trigger or button independent from the trigger 65. In some implementations, the clutch gear 134 and the worm gear 133 can be manually disengaged from one another through manual actuation of a trigger button. FIG. 3M shows a cross-sectional view of the drivetrain 120 housed in the main body 15. The drivetrain 120 includes the clutch gear 134 engaged with the worm gear 133, and the clutch gear 134 and the worm gear 133 can be engaged and disengaged as described with respect to FIGS. 3G to 3L. In the drivetrain 120 depicted in FIG. 3M, additionally or alternatively, the clutch gear 134 and the worm gear 133 can be engaged and disengaged from one another through manual actuation of a trigger button 166. The trigger button 166 projects from an opening in the main body 15 and is centered on the axis of the shaft 135 carrying the clutch gear 134. When a user presses the trigger button 166, the force on the trigger button 166 is transferred to the shaft 135, causing the shaft 135 to translate axially. The clutch gear 134 translates with the shaft 135 and thus disengages from the worm gear 133. The worm drive 130 is therefore disengaged from the carriage, enabling the user to manually reposition the carriage relative to the housing. The user can press the trigger button 166 and the shaft 135 against the spring force of the spring 138, which biases the shaft 135 such that the clutch gear 134 is biased back toward engagement with the worm gear 133 when the user releases the force on the trigger button 166.

In some implementations, the user can manually reposition the carriage relative to the housing only when the trigger button 166 is depressed. In some cases, the trigger button 166, when pressed, remains in the depressed position until the user applies a subsequent force on the button. In such cases, the user can reposition the carriage relative to the housing without having to hold onto the trigger button 166. For example, the trigger button 166 may include a latching mechanism that causes the trigger button 166 to latch into the main body 15 when the trigger button 166 is first depressed and the clutch gear 134 is disengaged. When the trigger button 166 is pressed again, the trigger button 166 is unlatched, and the spring 138 is able to push the trigger button 166 and the shaft 135 back such that the clutch gear 134 engages with the worm gear 133.

In some implementations, an additional spring can be positioned between the trigger button 166 and the main body 15. The spring can increase an amount of force that the user is required to exert on the trigger button 166. The increased amount of force can reduce the risk of inadvertent actuation of the trigger button 166. In some cases, instead of the additional spring, the trigger button 166 can actuate one or more mechanical linkages that reduce the amount of force required to cause the disengagement of the clutch gear 134 from the worm gear 133. The reduced amount of force can increase the ease at which the user can disengage the clutch gear 134.

In some implementations, the drivetrain 120 can include both the mechanical mechanism for disengagement of the clutch gear 134 from the worm gear 133 described with respect to FIG. M as well as the electromechanical mechanism for disengagement of the clutch gear 134 from the worm gear described with respect to FIGS. 3G to 3L. In some cases, the drivetrain 120 can include only the mechanical mechanism facilitated by the trigger button 166 or only the electromechanical mechanism facilitated by the motor 125 and the trigger 65. In some cases, the mechanism for the trigger button 166 and the mechanism for the trigger 65 are both mechanical.

FIGS. 3N to 3P show the syringe body grip mechanism 170 of the main body 15 where the syringe body would be placed. The syringe body grip mechanism 170 includes spring-loaded grips 175A, 175B that can be manually adjusted to grip syringes of a variety of diameters. FIG. 3O shows the portion of the syringe body grip mechanism 170 underlying grip 175A. Referring briefly to FIG. 3O, the grip 175A rotates about grip pivot 177A on the underside of the syringe body grip mechanism 170. Referring back to FIG. 3F, the syringe body grip mechanism 170 further includes a spring-loaded grip flange 180 that prevents the syringe body from sliding axially. Referring to FIG. 3O, the grip 175A is attached to torsion spring 181A which rotates the grip 175A toward grip 175B. Grip 175B also is attached to a torsion spring that rotates the grip 175B about another grip pivot towards grip 175A. Referring to FIG. 3P, the flange 180 is attached to a linear spring 185 that pulls the flange 180 against the loaded syringe body.

In some implementations, a rotary encoder may be attached to the grip pivots and/or a linear encoder may be attached to the linear spring to verify that the syringe has been correctly loaded or to determine the size of the syringe being used. In some implementations, the spring-loaded grips may include features to increase friction with the syringe. For example, the grips may include elastomeric surfaces where they contact the syringe to increase friction. The grips may also include suction features to keep the syringe from moving during use.

FIGS. 3D and 4 show the interface between the carriage 20 and the main body 15. Specifically, FIG. 4 shows a front cross-sectional view taken along a section line forward of a v-profile roller 35. FIG. 4 shows the interface between the v-profile roller 35 and a v-profile track 95. The carriage 20 travels on the eight v-profile rollers 35 (only one of which is shown in FIG. 4) that move along one or more v-profile tracks 95 (also referred to as c-profile tracks) that are molded into the main body 15. The v-profile rollers 35 and v-profile tracks 95 are asymmetric, having a steeper surface 36 (i.e., a surface with a higher angle of incline as compared to the other surface of the v-profile) on the side of the v-profile closer to the carriage frame 22. The steeper surface 36 can support side loads on the carriage 20 due to uneven loading or rough handling. The lower-angled outer surface 37 of the v-profile supports perpendicular forces due to the moments of the off-axis loads of the syringe plunger and gear rack. Referring to FIG. 3D, the rack-and-pinion mechanism and the drivetrain 120 allows rotational motion of the motor to induce a linear motion of the carriage 20. The pinion gear 100 is fixed in the main body and the rack 40 is on the underside of the moving carriage. The pinion gear 100 drives the rack 40 to move the carriage 20 forward towards the main body 15.

While the implementation as described above has eight v-profile rollers, the number and configuration of the v-profile rollers can vary. For example, the v-profile rollers could be on the main body while the one or more tracks for the v-profile rollers are disposed on the carriage. Other implementations may have fewer or more v-profile rollers, e.g., 2, 3, 4, or 5 v-profile rollers on each side of the carriage. The axles of the v-profile rollers may also include a bearing to further reduce friction in the system. While the rollers have been described as v-profile rollers, in some implementations, the rollers could have other profiles, such as an H shape or U shape.

### Electrical Connections of the Elements of the Syringe Pumps

FIG. 5 is a block diagram illustrating an example interconnection of electrical components of the syringe pump. In some implementations, at least some of the components may be included on a single printed circuited board that includes, for example, a microcontroller, voltage regulators, motor driver components, display driver components, and wireless communication components. The syringe pump includes a central processor 200 that receives inputs from a display 25 with a touch screen 205, a keypad input 210, an accelerometer 212 attached to the display 25, an external device 215 such as a personal computer or tablet device via a Bluetooth transceiver 217, a memory storage element 220, analog input 222, and a delivery processor 230 operable with an encoder 235 and a motor control 237 to control the motor 125. The central processor 200 delivers outputs to the display 25, the external device 215, the memory storage element 220, and the delivery processor 230.

In some implementations, the functionalities of the delivery processor 230 may be implemented using the central processor 200. While a Bluetooth transceiver 217 is described, any means of wired or wireless communication with an external device can be used to transfer information to and from the central processor. For example, a wireless transceiver such as a WiFi transceiver, or an external transceiver connected to a port (e.g., a Universal Serial Bus (USB) port) may also be used. In addition, while an external device is shown, the syringe pump may be operable without an external device. The components may also include additional mechanical position encoders and sensors connected to the central processor 200. For example, rotary encoders attached to the syringe body grips may help to determine the size of the syringe being placed into the syringe pump. A sensor, such as an optical detector, may be used to detect the fluid level or content of the syringe. The central processor may also receive force measurements from the force sensor on the pusher assembly to determine whether the syringe is properly seated in the pusher assembly.

In some implementations, the force measurements can be used to adjust the motor speed to compensate for changes in forces. For example, to compensate for backlash and other compliance that may add to imprecision of the gear train, the central processor can deliver instructions to the delivery processor to adjust the motor speed or to momentarily reverse direction of the motor. In some implementations, the central processor may be further programmed to respond to increases in flow or changes in pressure from, for example, a change in height between the pump and patient or a downstream blockage or occlusion. The central processor can be programmed to recognize an increase in pressure, such as may be caused by a downstream occlusion of the outflow tubing, beyond an expected value and stop or reverse the motor until the pressure has returned to normal limits. The controller can also be programmed to recognize a drop in measured force or an irregular motion of the driving mechanism and respond by momentarily reversing the motor direction. The controller may also raise an alarm or other signal to indicate the problem that has occurred.

A user can interact with the display 25 to give commands to the syringe pump, such as inputting a desired amount of fluid delivery or a duration of delivery. The keypad input 210 has an on/off switch and an emergency stop switch that allows a user to override other instructions from the central processor 200. The accelerometer 212 on the display 25 determines the position of the display 25 and can adjust the visual format of information shown on the display 25 depending on the orientation of accelerometer 212. For example, if the user rotates the display 25 sideways, the accelerometer 212 can detect the change in orientation and modify the display 25 so that the user can see information displayed in a convenient orientation.

The memory storage element 220 can include default settings for the syringe pump and serves a location for data files that have been uploaded to the central processor by an external device 215. The memory storage element 220 may further function as an archive for future uploading to an external device 215. These data files may include, for example, customized libraries of the characteristics of medical fluids and drugs, or particular infusion orders for a particular patient, generic protocols for specific types of infusion, such as patient controlled analgesia, epidural infusion, intermittent infusions, and/or error logs or usage tracking logs that the pump may archive for upload to an external source for forensic analysis, review, or continuous improvement. The user may be able to change these default settings via the display 25 or via the external device 215 or to transfer data of the types enumerated above. The external device 215 can collect data received by the central processor 200 or serve as an additional means for the user to monitor the syringe pump. The encoder 235 is operable with the syringe body grip mechanism and determines the size of the syringe being used. The motor 125 can transmit data related to its position and sense. The encoder 235 and the drive motor 125 are operable with a delivery processor 230. The drive motor 125 can be operable with the delivery processor 230 through the motor control 237.

### Computer System

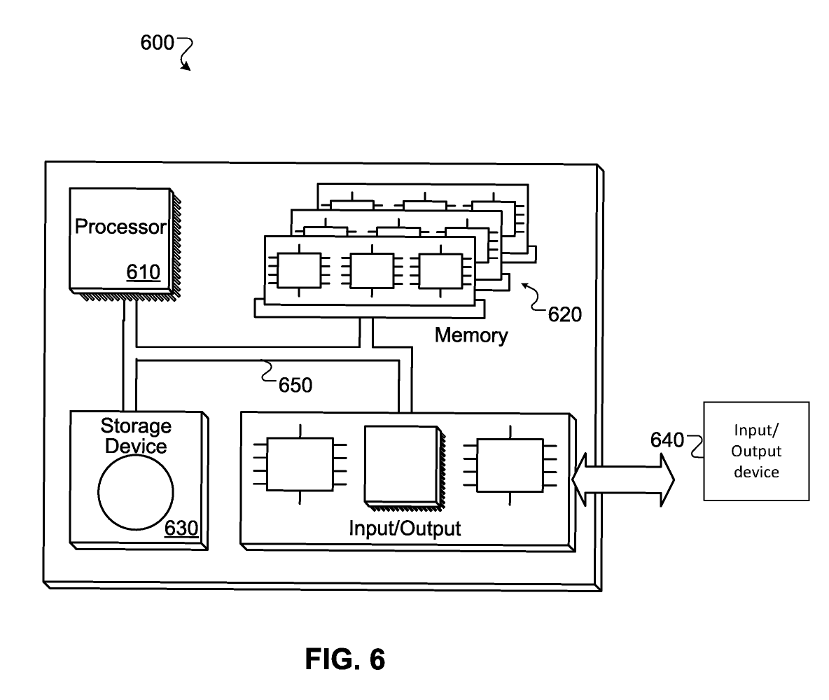

FIG. 6 is a schematic diagram of a computer system 600 at least a portion of which can be used for implementing the computing device that includes the touchscreen display 25 and the keypad 210. Portions of the computing system 600 described herein can be implemented into, for example, the central processor 200, the external device 215, and other computing devices of the electromechanical systems depicted in FIG. 5.

The computing system 600 can include, for example, a processor 610, a memory 620, a storage device 630, and an input/output device 640. Each of the components 610, 620, 630, and 640 are interconnected using a system bus 650. The processor 610 is capable of processing instructions for execution within the system 600. In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output device 640. The processor 610 can be operable with electrical and electromechanical components of the syringe pumps and syringe pump systems described herein.

The memory 620 stores information within the system 600. In some implementations, the memory 620 is a computer-readable medium. The memory 620 can include volatile memory and/or non-volatile memory.

The storage device 630 is capable of providing mass storage for the system 600. In one implementation, the storage device 630 is a computer-readable medium. In various different implementations, the storage device 630 may be a hard disk device, an optical disk device, or a solid state memory device. The memory 620 and/or the storage device 630 can store treatment parameters and parameters of the electromechanical systems of the syringe pumps described herein. These components can also store data collected by various sensors of the syringe pump, for example, data collected by the rotary encoder 235, the force sensor 60, the force sensor 99, or other appropriate sensors of the syringe pump. The memory 620 and/or the storage device 630 can also store data regarding the inputs (e.g., power input) into electromechanical components of the syringe pump, such as the motor 125. In some cases, the memory 620 and/or the storage device 630 can also store data pertaining to the progress of the treatment, such as the amount of fluid delivered or the duration of treatment that has elapsed.

The input/output device 640 provides input/output operations for the system 600. In some implementations, the input/output device 640 includes a keyboard (e.g., the keypad 210) and/or a pointing device. In some implementations, the input/output device 640 includes a display unit for displaying graphical user interfaces. In some implementations the input/output device can be configured to accept verbal (e.g., spoken) inputs. For example, the clinician can provide the input by speaking into the input device. The input/output device can also include a touchscreen display such as the display 25. The touchscreen display device may be, for example, a capacitive display device operable by touch, or a display that is configured to accept inputs via a stylus.

The features computing systems described herein can be implemented in digital electronic circuitry, or in computer hardware, firmware, or in combinations of these. The features can be implemented in a computer program product tangibly embodied in an information carrier, e.g., in a machine-readable storage device, for execution by a programmable processor; and features can be performed by a programmable processor executing a program of instructions to perform functions of the described implementations by operating on input data and generating output. The described features can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Computers include a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

The processor 610 carries out instructions related to a computer program. The processor 610 may include hardware such as logic gates, adders, multipliers and counters. The processor 610 may further include a separate arithmetic logic unit (ALU) that performs arithmetic and logical operations.

### Methods of Use

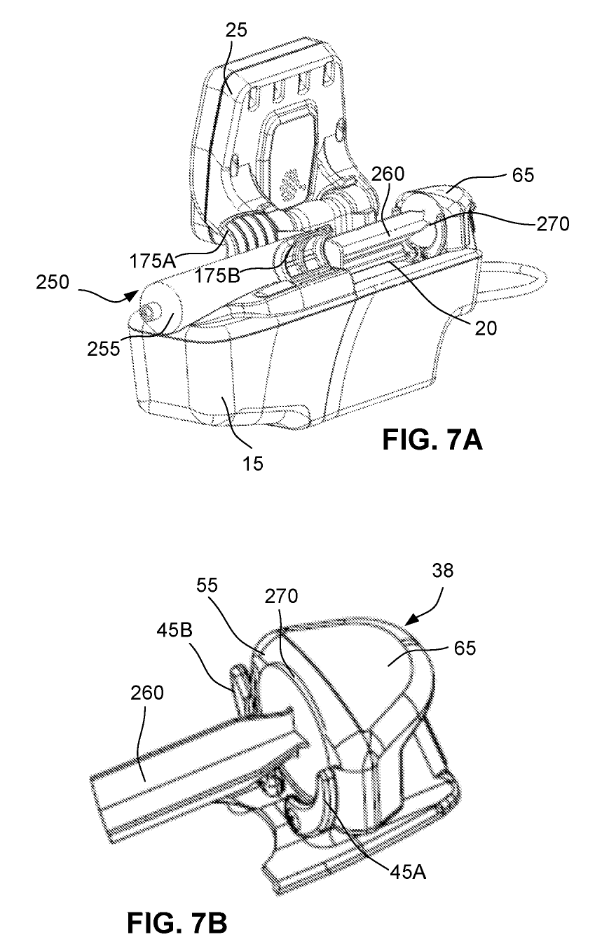

FIGS. 7A to 7B, 8A to 8B, and 9A to 9B depict an example of a method of using the syringe pumps described herein. Referring to FIG. 7A, the user loads a syringe 250 that includes a syringe body 255 and a syringe plunger 260 into the main body 15 and the carriage 20 of the syringe pump 10. The syringe body is loaded into the main body 15, and the syringe plunger is loaded into the carriage 20. The syringe pump 10 is intended for use in several orientations. It may be mounted horizontally on an IV pole with the display 25 and syringe 250 facing toward the user. The syringe pump 10 may also be used vertically on an IV pole or sitting on a table or other flat surface with the syringe 250 horizontal and the display facing upward.

Referring to FIG. 7B, prior to inserting the syringe 250, the user depresses the trigger 65 to release the clamp arms 45A, 45B. While depressing the trigger 65, the user loads the syringe 250 as shown in FIG. 7A and 7B by pushing the syringe body 255 past the spring-loaded grips 175A, 175B. The user also engages the flange 180 (shown in FIG. 3O) with the syringe body 255 so as to prevent the syringe 250 from moving axially. Upon releasing the trigger 65, the clamp arms 45A, 45B capture the plunger flange 270 of the syringe plunger 260 and hold the plunger flange 270 in place. The clamp arms 45A, 45B center and hold in place the syringe plunger 260. The forward surfaces of the plunger flange 270 press against a fixed stop and the rearward surface of the plunger flange 270 press against the pusher surface 55, which can be a spring-loaded plate. Axial movement of the syringe body 255 is thus prevented in both directions. When the plunger flange 270 presses against the pusher surface 55, it produces a force read by the force sensor. The syringe 250 is mounted so that the syringe body 255 cannot move relative to the main body 15 and the syringe plunger 260 is limited from moving relative to the carriage 20 and pusher assembly 38.

In some cases, when the user depresses the trigger 65, the clamp arms 45A, 45B also translate in the forward direction away from the pusher surface 55. The translation of the clamp arms 45A, 45B enable the clamp arms 45A, 45B and the pusher assembly to accommodate syringes with thicker plunger flanges.

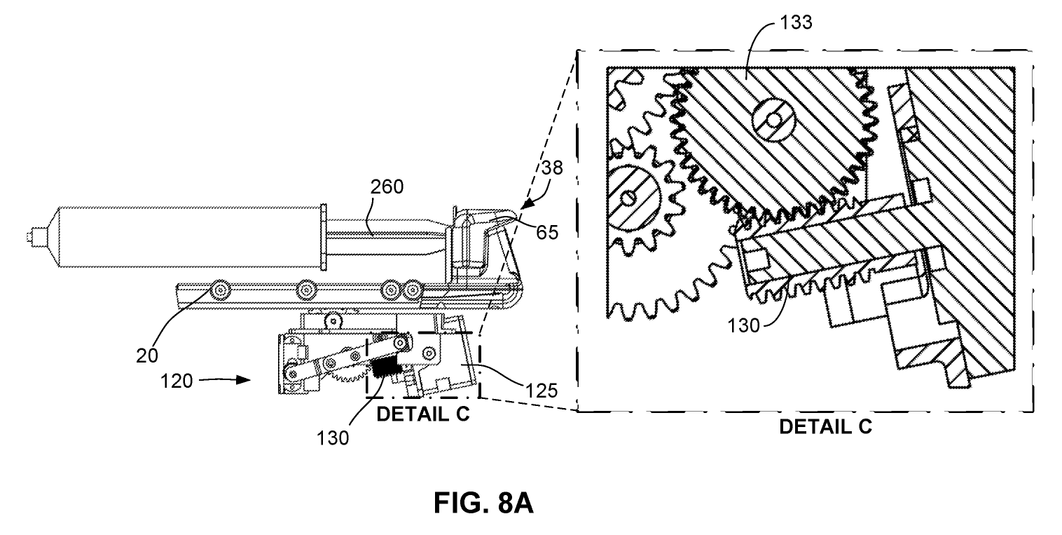

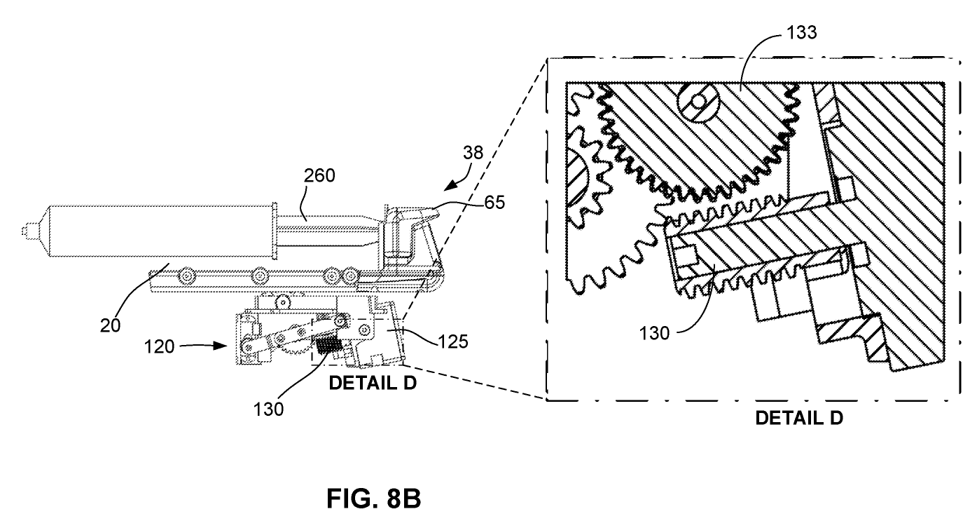

The worm gear engagement and disengagement mechanism (also referred to as a release mechanism) is provided to allow a user to manually reposition the carriage relative to main body. FIG. 8A shows the worm drive 130 engaged to the adjacent worm gear 133. In particular, Detail C of FIG. 8A further shows a side cross-sectional view of the drivetrain 120 with the worm drive 130 engaged with the worm gear 133. FIG. 8B shows the worm drive 130 disengaged from the adjacent worm gear. Detail D of FIG. 8B further shows a side cross-sectional view of the drivetrain 120 with the worm drive 130 disengaged from the worm gear 133.

In some implementations, the user continuously presses the trigger 65 on the pusher assembly 38 to release the worm drive 130. While the worm drive 130 is disengaged, the user may move the carriage 20 so that the syringe plunger 260 can be placed in a position ready for treatment. As soon as the user's finger is no longer engaging the trigger 65 on the pusher assembly 38, the worm drive 130 will re-engage. When the worm drive 130 is engaged, the user can no longer manually reposition the syringe plunger 260, because the worm gear 133 cannot be easily driven backwards. In case of power or other failure, the worm drive 130 is biased by a spring (not shown) that immediately moves the worm drive 130 back into mesh and prevent further carriage movement. Resistance in the drivetrain 120, from, for example, the worm drive 130, reduces the effect of external forces that can backdrive the drivetrains. As a result, the main body and carriage are essentially locked together whenever the drive motor 125 is not turning. Thus, free flow or siphoning is prevented when the motor 125 is paused or if power is lost.

FIGS. 8A to 8B show rotation of the worm drive 130 to disengage and engage the worm drive 130 from the drivetrain 120. In some implementations, as described with respect to FIGS. 3G to 3L, the worm drive 130 can additionally or alternatively be disengaged from and engaged to the drivetrain 120 by disengaging the clutch gear 134 from or engaging the clutch gear 134 with the worm gear 133. In particular, when the clutch gear 134 is engaged with the worm gear 133 (FIGS. 3I and 3J), the user can depress the trigger to activate the clutch mechanism to disengage the clutch gear 134 from the worm gear 133 (FIGS. 3K and 3L). The user can then manually reposition the carriage relative to the main body. When the user releases the trigger, the clutch gear 134, biased by the force of the spring 138, re-engages with the worm gear 133 (FIGS. 3I and 3J) without further intervention from the user.

With the worm drive 130 engaged and the syringe 250 loaded in the syringe pump, the syringe pump is ready for use. Referring back to FIG. 1, the user flips the display 25 into the operating position. The user may operate the touchscreen of the display 25 or the keypad 210 to turn the syringe pump 10 on or off. For treatment, the user turns on the syringe pump 10 and, after completion of suitable manual priming activity, begins delivery of a fluid, such as a drug solution, contained within the syringe 250. The syringe pump 10 drives its rack-and-pinion mechanism in a controlled manner to slowly move the carriage 20 carrying the syringe plunger 260 toward the main body 15 carrying the syringe body 255. Such a motion delivers the drug solution in the syringe 250 into a vein or artery, or muscle or other tissue, of the patient.

During operation, the syringe pump electrical hardware, as depicted by example in FIG. 5, may begin monitoring the various components of the system. For example, the absolute encoder (not shown) on the main body, which engages with the secondary tooth rack 44 (shown in FIG. 2C) on the bottom of the carriage, adjacent to rack 40, or on the drivetrain, detects whether the carriage 20 is moving as expected. Motor power can be shut off and an alarm raised if an error is detected. Similarly, sensors on the clamp arms 45A-B and the pusher surface 55 of the carriage can detect whether the syringe 250 is correctly mounted and if excess driving force is being applied. In case of error conditions motor power can be shut off and an alarm is raised, or other appropriate response to the fault condition.

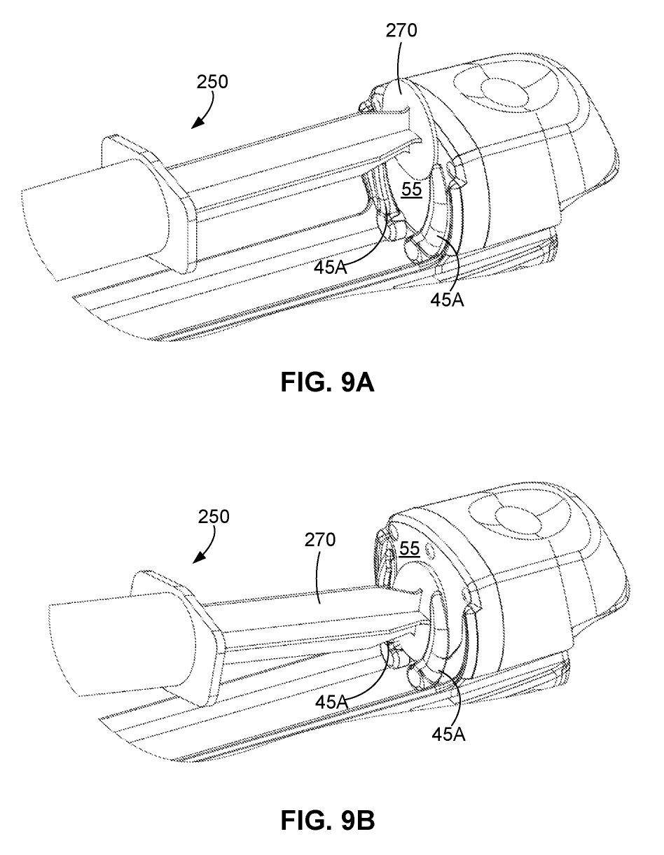

After the operation is complete, the user can remove the syringe 250 from the syringe pump 10. During the removal, the user can move the plunger flange 270 along the pusher surface 55, as shown in FIG. 9A. In some implementations, the user may rotate the plunger flange 270 relative to the pusher surface 55, as shown in FIG. 9B. As a result, the plunger flange 270 causes the clamp arms 45A, 45B to experience a force in the forward direction. As described with respect to FIGS. 2G to 2H, the clamp arms 45A, 45B, in some cases, are able to move relative to the pusher surface 55. Thus, the user can remove the plunger flange 270 with slight rotation of the plunger flange 270 relative to the longitudinal axis of the syringe 250 because the clamp arms 45A, 45B translate in the forward direction in response to the rotational motion. After the syringe 250 is removed, the retraction springs can cause the clamp arms 45A, 45B to retract back towards the pusher surface 55.

### OTHER EMBODIMENTS

It is to be understood that while the technology has been described in conjunction with the detailed description, the foregoing description and Examples are intended to illustrate and not limit the scope defined by the appended claims. Other aspects, advantages, and modifications are within the scope of the following claims.

What is claimed is:

1. An infusion apparatus adapted for use with a syringe, the apparatus comprising:
a housing having a drivetrain that includes a pinion, a plurality of spur gears, and a worm gear, wherein at least one of the spur gears is arranged to engage with the pinion and one of the spur gears is arranged to engage with the worm gear;
a carriage movable with respect to the housing and having a first side and a second side opposite the first side, wherein the carriage comprises:
a frame on the first side of the carriage, wherein the frame is configured to receive at least a portion of a plunger of the syringe,
a toothed rack disposed on the second side of the carriage, and extending along a longitudinal axis of the carriage, wherein the rack is configured to engage with the pinion for moving the carriage in a direction parallel to a longitudinal axis of the housing, and
a pusher assembly adapted to securely engage with the plunger of the syringe;
a motor arranged within the housing configured to rotate a worm drive that meshes with the worm gear to drive the drivetrain; and
a release mechanism comprising a release trigger configured to enable the worm drive to be disengaged from the worm gear, thereby enabling movement of the carriage with respect to the housing in a rearward direction.

2. The apparatus of claim 1, further comprising:
a secondary motor arranged within the housing configured to actuate the release mechanism such that the worm drive rotates from an engaged position in which the worm drive is engaged with the worm gear to a disengaged position in which the worm drive is disengaged from the worm gear.

3. The apparatus of claim 1, wherein the carriage further comprises a plurality of wheels that allow the carriage to move along tracks arranged on the housing, the tracks disposed along the longitudinal axis of the housing.

4. The apparatus of claim 3, wherein one or more of the tracks comprise an asymmetric v-profile track configured to accept one or more v-profile roller wheels of the carriage.

5. The apparatus of claim 4, wherein the asymmetric v-profile comprises two inclined surfaces joined along a line, and wherein an angle of incline of one of the surfaces is less than an angle of incline of the other surface.

6. The apparatus of claim 3, wherein the tracks are configured to accept the plurality of wheels such that a translation motion of the wheels in directions perpendicular to the direction parallel to a length of the rack is constrained.

7. The apparatus of claim 3, wherein a wheelbase corresponding to a pair of wheels is substantially equal to one half of a maximum distance traveled by the carriage.

8. The apparatus of claim 1, wherein the frame of the carriage is constructed of material comprising a fiber-reinforced plastic composite.

9. The apparatus of claim 1, wherein the motor is a stepper motor.

10. The apparatus of claim 1, wherein one or more of the spur gears are configured to prevent backlash effects.

11. The apparatus of claim 1, wherein the frame further comprises a pair of hinged arms configured to hold a body of the syringe onto the frame.

12. The apparatus of claim 11, wherein the hinged arms are convex in shape, and counter sprung towards one another.

13. The apparatus of claim 11, wherein the hinged arms are configured to hold the syringe on the frame such that a longitudinal axis of the plunger is aligned to a center of the pusher assembly.

14. The apparatus of claim 11, wherein the hinged arms are movable relative to the pusher assembly in a direction parallel to the longitudinal axis of the carriage.

15. The apparatus of claim 14, wherein the frame further comprises a spring assembly configured to apply a force on each of the hinged arms along the longitudinal axis of the carriage toward the pusher assembly.

16. The apparatus of claim 1, further comprising a display device operable to display one or more parameters related to a fluid delivered using the infusion apparatus.

17. The apparatus of claim 16, wherein the display device is attached to the housing via one or more hinges.

18. The apparatus of claim 17, wherein the one or more hinges are positioned to prevent the display device from covering at least a portion of a body of the syringe.

19. The apparatus of claim 16, wherein the display device is configured to accept user input related to an operation of the apparatus.

20. The apparatus of claim 1, further comprising one or more processing devices configured to control operations of the motor.

21. The apparatus of claim 20, further comprising at least one force sensor configured to provide a feedback signal to the one or more processing devices.

22. The apparatus of claim 21, wherein the one or more processing devices are configured to generate a control signal to adjust a speed or a direction of the motor in response to the feedback signal.

23. The apparatus of claim 21, wherein the at least one force sensor comprises a force sensor configured to measure a force exerted by the pusher assembly on the plunger.

24. A method of dispensing a fluid from a syringe disposed on an infusion pump, the method comprising:
engaging a motor with a drivetrain using a release mechanism configured to enable a worm drive to be engaged to a worm gear of the drivetrain; and
controlling a movement of a plunger of the syringe through a body of the syringe using the drivetrain, the drivetrain including a pinion, a plurality of spur gears and the worm gear, wherein at least one of the spur gears is arranged to engage with the pinion and one of the spur gears is arranged to engage with the worm gear, and
wherein the plunger is disposed on a carriage having a first side and a second side opposite the first side, wherein the carriage comprises:
a frame on the first side of the carriage, configured to receive at least a portion of the plunger of the syringe,
a toothed rack disposed on the second side of the carriage, and extending along a longitudinal axis of the carriage, wherein the rack is configured to engage with the pinion for moving the carriage in a direction parallel to the longitudinal axis of the carriage, and
a pusher assembly adapted to securely engage with the plunger of the syringe.

25. An infusion apparatus adapted for use with a syringe, the apparatus comprising:
a housing having a drivetrain that includes a pinion, a plurality of spur gears, the plurality of spur gears comprising a first clutch gear, a second clutch gear arranged to engage with the first clutch gear, and a spur gear arranged to engage with the pinion;
a carriage movable with respect to the housing and having a first side and a second side opposite the first side, wherein the carriage includes:
a frame on the first side of the carriage, wherein the frame is configured to receive at least a portion of a plunger of the syringe,
a toothed rack disposed on the second side of the carriage, and extending along a longitudinal axis of the carriage, wherein the rack is configured to engage with the pinion for moving the carriage in a direction parallel to a longitudinal axis of the housing, and
a pusher assembly adapted to securely engage with the plunger of the syringe;
a motor arranged within the housing configured to rotate a worm drive to drive the drivetrain; and
a release mechanism including a release trigger configured to disengage the second clutch gear from the first clutch gear, thereby enabling movement of the carriage with respect to the housing, the movement being independent of a motion of the worm drive.

26. The apparatus of claim 25, further comprising:
a secondary motor arranged within the housing, the secondary motor configured to actuate the release mechanism to translate the first clutch gear from a first position in which the first clutch gear is engaged to the second clutch gear to a second position in which the first clutch gear is disengaged from the second clutch gear.

27. The apparatus of claim 26, wherein
the first clutch gear is a male clutch gear,
the second clutch gear is a female clutch gear, and
the male clutch gear, in the first position, engages inner teeth of the female clutch gear.

28. The apparatus of claim 25, further comprising:
a trigger button movable relative to the housing and configured to actuate the release mechanism such that the first clutch gear translates from a first position in which the first clutch gear is engaged to the second clutch gear to a second position in which the first clutch gear is disengaged from the second clutch gear.
